(19) United States
(12) Patent Application Publication
(10) Pub. No.: US 2009/0130675 A1
(43) Pub. Date: May 21, 2009

(54) Genes Involved in the Biosynthesis of Thiocoraline and Heterologous Production of Same

(75) Inventors: Angelina Ramos Castro, Oviedo (ES); Alfredo Fernandez Brana, Oviedo (ES); Felipe Lombo Brugos, Oviedo (ES); Carmen Mendez Fernandez, Oviedo (ES); Jose Antonio Salas Fernandez, Oviedo (ES); Ana Velasco Iglesias, Madrid (ES); Carmen Schleissner Sanchez, Madrid (ES)

Correspondence Address: KING & SPALDING, 1185 AVENUE OF THE AMERICAS, NEW YORK, NY, 10036-4003, US

(73) Assignee: PHARMA MAR, S.A., Madrid (ES)

(21) Appl. No.: 11/997,692

(22) PCT Filed: Aug. 1, 2006
(86) PCT No.: PCT/ES06/00455

§ 371 Date: Apr. 7, 2008

(30) Foreign Application Priority Data
Aug. 2, 2005 (ES) P200501932

(51) Int. Cl. C12Q 1/68; C12N 15/11; C07H 21/04; C12N 15/00; C12N 1/21; C07K 14/00; C12P 21/04; C12P 17/16

(52) U.S. Cl. 435/6; 536/23.7; 536/24.3; 435/320.1; 435/252.3; 435/252.35; 530/350; 435/71.1; 435/118

(57) ABSTRACT

The invention relates to genes involved in the biosynthesis of thiocoraline and to the heterologous production of same. According to the invention, the cluster of genes responsible for the biosynthesis of thiocoraline was identified and cloned. Said cluster of genes can be used in the heterologous production of thiocoraline which has an antitumor and antibacterial activity.

This application is the entry of the national phase under 371 of PCT/ES2006/000455, filed Aug. 1, 2006, which claims foreign priority to ES P200501932, filed Aug. 2, 2005, the contents of each of which are incorporated by reference.

### FIELD OF THE INVENTION

The present invention relates to the cluster of genes responsible for the biosynthesis of thiocoraline and its use in the heterologous production of thiocoraline.

### BACKGROUND OF THE INVENTION

Thiocoraline (I)

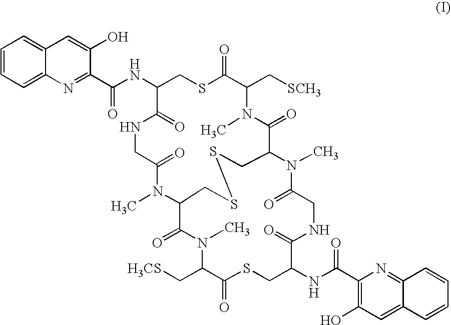

is a cyclodimeric thiodepsipeptide isolated from a marine actinomycete, specifically from the Micromonosporaceae family (Pérez Baz et al., J. Antibiotics, 50(9), 738-741, 1997; Romero et al., J. Antibiotics, 50(9), 734-737, 1997). Although it has been described that thiocoraline is obtained from Micromonospora marina or Micromonospora sp. L-13-ACM-092, subsequent studies have shown that the compound can also be isolated from the actinomycete Micromonospora sp. ML1, which was isolated from a marine mollusk found on the Indian ocean coast, in Mozambique (Espliego, F. Ph.D. Thesis, 1996, University of Leon; de la Calle, F. Ph.D. Thesis, 1998, Autonomous University of Madrid).

In vitro studies have shown the capacity of thiocoraline to inhibit the growth of cell lines of different types of solid tumors, such as melanoma, breast, non-microcytic lung and colon cancer. Thiocoraline has also shown that it has a marked antitumor activity in in vivo assays against human carcinoma xenografts (Faircloth et al. Eur. J. Cancer, 33, 175, 1997 (abstract)). Thiocoraline further shows antibacterial activity against Gram-positive bacteria.

Although obtaining thiocoraline from said marine actinomycete (Micromonospora sp. ML1) is feasible on a small scale, on a large scale said obtainment is limited due to the variability in the production that is observed with this microorganism. Indeed, the production of thiocoraline from said organism is a time-consuming process due to the low growth rate of this organism and shows important fluctuations in the production yields in different batches.

Therefore, due to the fact that on one hand, obtaining thiocoraline from said marine actinomycete is quite limited, and on the other hand, the fact that the thiocoraline molecule also has a complex structure and its synthesis can be complicated on an industrial level, it is desirable to understand the genetic bases of its biosynthesis for the purpose of creating means for affecting its obtainment in a directed manner. This could give rise to an increase in the amounts of thiocoraline produced, given that natural producing strains generally produce the product at low concentration and in a very irregular manner. Likewise, it could also allow the production of thiocoraline in hosts that do not produce this compound naturally.

The development of recombinant DNA technology has opened up an interesting field of research for generating and producing bioactive compounds by means of manipulating genes involved in the biosynthesis of such bioactive compounds, mainly of bacteria from the actinomycete group. These techniques can be used to improve the production of already known natural compounds, because natural strains usually produce low concentrations of the metabolite of interest.

The heterologous expression of the cluster of genes involved in the biosynthesis of thiocoraline in other actinomycetes that are more suitable for genetic manipulation and fermentation would likewise allow producing said compounds with more reproducible yields in shorter fermentation times.

As is known, a number of bacteria and fungi synthesize a wide variety of biologically active peptides with a nonribosomal origin, including antitumor and antibacterial peptides, etc. The biosynthesis of this family of compounds is carried out by nonribosomal peptide synthetases (NRPSs), which are multifunctional enzymes with a modular catalytic domain organization. Each of these modules carries out an elongation cycle, i.e., it activates and incorporates a specific amino acid into the final structure of the compound. A minimal module is formed by three domains: (i) an adenylation domain, (A, with approximately 550 amino acids) which is responsible for selecting a certain amino acid and generating the adenylated aminoacyl version thereof by means of using ATP; (ii) a peptidyl carrier domain (P, with approximately 80 amino acids) containing a phosphopantetheine (PP) prosthetic group acting as cofactor and binding to the P domain by a covalent bond; this domain is responsible for fixing the activated adenylated amino acid before passing to the following reaction centers; and (iii) a condensation domain (C, with approximately 450 amino acids) generating a new peptide bond between two adenylated aminoacyl moieties located in two consecutive P domains. C domains are absent in the modules activating the first amino acid of the system. Some NRPSs have extra domains for carrying out specific activities, such as epimerizations giving rise to D-amino acids, N- or C-type methylations, circularizations acting on the L-Cys or L-Ser amino acids. A final domain located after the last module, is generally responsible for releasing the intermediate enzyme, generating a linear or cyclic peptide. As a general rule, the structure of the different modules reflects the final amino acid sequence of the product peptide. This colinearity rule allows assigning a specific activation function to each module in an NRPS. Information on NRPSs can be found, for example, in Quing-Tao, S. et al., 2004. Dissecting and Exploiting Nonribosomal Peptide Synthetases. Acta Biochimica et Biophysica. Sinica, 36 (4): 243-249.

### SUMMARY OF THE INVENTION

An important objective of the present invention consists of isolating and characterizing the complete nucleotide sequence encoding the proteins responsible for the production of thiocoraline. Based on this, the function of the amino acid sequences comprising the proteins involved in the biosynthesis of thiocoraline can be isolated and determined. This objective can be reached by providing an isolated and optionally purified new nucleic acid molecule encoding all the proteins related to the complete biosynthetic thiocoraline production pathway.

The inventors have been able to identify and clone all the genes responsible for the biosynthesis of thiocoraline, i.e., the cluster of genes involved in the biosynthesis of thiocoraline, providing the genetic bases for improving an manipulating the production of this compound in a directed manner.

By means of using initiator oligonucleotides derived from consensus sequences of nonribosomal peptide synthetase (NRPS) adenylation domains, 6 fragments of Micromonospora sp. ML1 chromosome were amplified by means of the polymerase chain reaction (PCR), all of which fragments contain putative (hypothetical) NRPS adenylation domain fragments called PSV1, PSV2, PSV3, PSV4, PSV5 and PSV6 (Example 3). The inactivation, by insertion, of said adenylation domains has shown that two of them (PSV2 and PSV5) generated mutants that do not produce thiocoraline, which indicated that they were involved in the biosynthesis of thiocoraline (Examples 7 and 10).

The sequencing of a DNA region of approximately 64.6 kilobases (kb) (SEQ ID NO: 1) showed the presence of 36 complete open reading frames (ORFs) and another 2 incomplete ORFs (Example 12, Table 1). The heterologous expression of a region of approximately 53 kb, containing 26 of said ORFs, in Streptomyces coelicolor, Streptomyces albus and Streptomyces lividans led to the production of thiocoraline in said actinomycetes (Example 19).

The cluster of genes responsible for the biosynthesis of thiocoraline is schematically shown in FIG. 1. Surprisingly enough, the cluster of thiocoraline genes contains more NRPS encoding genes than those expected based on the number of amino acids of the peptide skeleton. Some of the identified proteins are involved in the formation of the thiocoraline peptide structure, such as several of the NRPSs identified as Tio12, Tio17, Tio18, Tio19, Tio20, Tio21, Tio22, Tio27 and Tio28, for example. The proteins identified as Tio20 and Tio21 probably form the NRPSs involved in the biosynthesis of the thiocoraline skeleton and probably, other two NRPSs, identified as Tio27 and Tio 28 could be responsible for the biosynthesis of a small peptide which could be involved in regulating the biosynthesis of thiocoraline in Micromonospora sp. ML1. There are also several proteins which could be related to resistance process, such as Tio5, Tio6 and Tio23. The possible regulators of the thiocoraline pathway identified in the sequenced region correspond to Tio3, Tio4, Tio7, Tio24 and Tio25. Finally, there are also several proteins related to the generation of the initiator unit 3-hydroxy-quinaldate, Tio8, Tio9, Tio10 and Tio1. The genes, the gene interruption of which generates a phenotype that does not produce thiocoraline, are indicated in FIG. 1 by means of an asterisk (tio20, tio27 and tio28).

The present invention therefore relates to the identification and cloning of the cluster of genes responsible for the biosynthesis of thiocoraline. Said cluster of genes responsible for the biosynthesis of thiocoraline and its expression in a suitable host cell allows the efficient production of thiocoraline.

Consequently, in one aspect, the invention relates to an isolated nucleic acid molecule comprising a nucleotide sequence encoding at least one biosynthetic thiocoraline production pathway protein, or a biologically active fragment thereof.

In another aspect, the invention relates to a composition comprising at least one nucleic acid molecule provided by this invention.

In another aspect, the invention relates to a probe comprising a nucleic acid molecule provided by this invention or a fragment thereof.

In another aspect, the invention relates to a vector comprising a nucleic acid molecule provided by this invention.

In another aspect, the invention relates to a host cell transformed or transfected with a vector provided by this invention.

In another aspect, the invention relates to a protein encoded by a nucleic acid molecule provided by this invention.

In another aspect, the invention relates to a method for producing a protein involved in the biosynthesis of thiocoraline, comprising the use of a thiocoraline-producing organism the genome of which has been manipulated.

In another aspect, the invention relates to a process, based on the use of genes responsible for the biosynthesis of thiocoraline from Micromonospora sp. ML1, for the production of thiocoraline in another actinomycete.

### DESCRIPTION OF THE DRAWINGS

FIG. 1. Schematic depiction of the cluster of thiocoraline genes and of the genes surrounding them, including the gene organization of the sequenced Micromonospora sp. ML1 chromosome area. The restriction sites used to construct the plasmids for the heterologous expression of the cluster of thiocoraline genes are shown.

FIG. 2. Schematic depiction of the cosmids cosV33-D12 and pCT2c. ori: replication origin for E. coli. SCP2: replication origin for Streptomyces. aac(3)IV: apramycin resistance gene. neo: neomycin resistance gene. bla: ampicillin resistance gene. SV40 ori: eukaryotic origin for episomal replication.

FIG. 3. Diagram of clonings carried out for constructing plasmid pFL1036. ori: replication origin for E. coli. M13 ori: replication origin for the M13 phage. oriT: conjugative transfer origin. lacZ: beta-galactosidase gene. kan^{R}: kanamycin resistance gene. aac(3)IV: apramycin resistance gene. bla: ampicillin resistance gene.

FIG. 4. Diagram of clonings carried out for constructing plasmid pFL1041. ori: replication origin for E. coli. SCP2: replication origin for Streptomyces. oriT: conjugative transfer origin. lacZ: beta-galactosidase gene. aac(3)IV: apramycin resistance gene.

FIG. 5. Diagram of clonings carried out for constructing plasmid pAR15AT. ori p15A: replication origin for E. coli. oriT: conjugative transfer origin. intφC31: φC31 phage integrase gene. attP: site-specific recombination site. kan^{R}: kanamycin resistance gene. aac(3)IV: apramycin resistance gene.^{K}: cleavage site treated with the Klenow fragment of the E. coli DNA polymerase.

FIG. 6. Diagram of clonings carried out for constructing plasmid pAPR. ori p15A: replication origin for E. coli. oriT: conjugative transfer origin. ori M13: replication origin of the M13 phage. ori: replication origin for E. coli. lacZ: beta-galactosidase gene. lacI: lactose operon repressor gene. intφC31: φC31 phage integrase gene. attP: site-specific recombination site. kan^{R}: kanamycin resistance gene. aac(3)IV: apramycin resistance gene.^{K}: cleavage site treated with the Klenow fragment of E. coli DNA polymerase. P_{ermE}: ermE gene promoter.

FIG. 7. Depiction of plasmids pFL1048, pFL1048r and pFL1049. ori p15A: replication origin for E. coli. oriT: conjugative transfer origin. intφC31: φC31 phage integrase gene. attP: site-specific recombination site. aac(3)IV: apramycin resistance gene.

FIG. 8A. HPLC chromatogram of a Streptomyces albus (pFL1049) culture extract after 7 days of growth in R5A medium. The peak corresponding to thiocoraline and its retention time, 27 minutes, are shown.

FIG. 8B. UV absorption spectrum of the product (thiocoraline) present in the peak of 27 minutes shown in FIG. 8A.

FIG. 8C. Mass spectrum of the product (thiocoraline) present in the peak of 27 minutes shown in FIG. 8A.

### DETAILED DESCRIPTION OF THE INVENTION

According to the present invention, a new, isolated and optionally purified nucleic acid molecule encoding all or part of the proteins involved in the complete biosynthetic thiocoraline production pathway is provided.

Therefore, in one aspect, the invention relates to a nucleic acid molecule, hereinafter, nucleic acid molecule of the invention, preferably an optionally purified, isolated nucleic acid molecule comprising a nucleotide sequence encoding at least one biosynthetic thiocoraline production pathway protein, or a biologically active fragment thereof. Said biosynthetic thiocoraline production pathway protein is generally a nonribosomal peptide synthetase (NRPS). NRPSs are responsible for the biosynthesis of thiocoraline.

As used herein, the expression “biologically active fragment”, applied to a biosynthetic thiocoraline production pathway protein, relates to a part of the protein structure retaining the active function of the full-length protein. Said biologically active fragments can be encoded by the corresponding regions of the nucleic acid molecule of the invention. The size of said regions of the nucleic acid molecule of the invention can vary within a wide range; nevertheless, in one particular embodiment, said regions can have a length of at least 10, 15, 20, 25, 50, 100, 1,000, 2,500, 5,000, 10,000, 20,000, 25,000 or more nucleotides. Said regions normally have a length between 100 and 10,000 nucleotides, preferably between 100 and 7,500, and are biologically functional, i.e., they can encode a biologically active fragment of a biosynthetic thiocoraline production pathway protein.

The nucleic acid molecule of the invention can be a deoxyribonucleic acid (DNA) or ribonucleic acid (RNA) molecule. The nucleic acid molecule of the invention can also be a single-strand nucleic acid molecule or a derived double-strand nucleic acid molecule. Illustrative non-limiting examples of nucleic acid molecules of the invention include genomic DNA (gDNA) molecules, messenger RNA (mRNA) molecules and complementary DNA (cDNA) molecules to mRNA molecules.

The mutants and variants of the nucleic acid molecule of the invention are included within the scope of the present invention. Said mutants and variants include the nucleic acid molecules of the invention in which at least one molecule has been altered, substituted, eliminated or inserted. By way of illustration, the mutants and variants of the nucleic acid molecule of the invention can have 1, 2, 3, 4, 5, 10, 15, 25, 50, 100, 200, 500 and more changes (alterations, substitutions, eliminations or insertions) of nucleotides. Degenerate variants encoding the same protein, as well as non-degenerate variants encoding a different protein are also possible. The nucleotide sequence of said mutants and variants encodes a protein, or a biologically active fragment thereof, conserving at least one of the biological activities or functions of the corresponding protein encoded by any open reading frame (ORF) of the cluster of genes responsible for the biosynthesis of thiocoraline. The allelic forms of the genes of said cluster as well as the polymorphisms are also comprised within the scope of the present invention.

In one particular embodiment, the nucleic acid molecule of the invention is an optionally purified, isolated nucleic acid molecule comprising a nucleotide sequence encoding all the biosynthetic thiocoraline production pathway proteins, or biologically active fragments thereof. In this case, the nucleic acid molecule of the invention comprises the nucleotide sequence containing the complete cluster of genes responsible for the biosynthesis of thiocoraline.

The nucleotide sequence of the complete cluster of genes responsible for the biosynthesis of thiocoraline is included in SEQ ID NO: 1, a 64,650 base pair (bp) genomic DNA sequence of Micromonospora sp. ML1. The scope of the invention also includes the complementary strand to the nucleotide sequence shown in SEQ ID NO: 1, i.e., that formed by nucleotides which are complementary to those indicated in SEQ ID NO: 1 (e.g., A substituted with T, C substituted with G and vice versa) and/or reverse nucleotide sequences [i.e., the sequences generated by changing the reading direction e.g., from (5′→3′) to (3′→5′)].

The present invention further includes a nucleic acid molecule hybridizing with the nucleic acid molecule of the invention having the nucleotide sequence shown in SEQ ID NO: 1 or its complementary strand; said molecule can be isolated from a thiocoraline-producing organism and encodes at least one biosynthetic thiocoraline production pathway protein. Typical hybridization techniques and conditions, known by persons skilled in the art, are mentioned, for example, in Sambrook et al., Molecular Cloning: A Laboratory Manual, Second Edition, Cold Spring Harbor Laboratory Press, Cold Spring Harbor, N.Y. (1989). Conventional or severe hybridization techniques are generally used for homologous probes, whereas less severe hybridization conditions are used for partially homologous probes having less than 100% of homology with the target nucleic acid molecule sequence. In the latter case (partially homologous probes), a series of Southern or Northern hybridizations with different conditions can be carried out. By way of illustration, when hybridization is carried out in a solvent containing formamide, the preferred conditions include the use of a constant temperature and ionic strength of approximately 42° C. with a solution containing 6×SSC, 50% of formamide. Less severe hybridization conditions can use the same temperature and ionic strength although in this case, the amount of formamide in the annealing buffer will be lower (from approximately 45% to 0%). Alternatively, hybridization can be carried out in aqueous solutions that do not contain formamide. In general, for the hybridization in aqueous medium, the ionic strength of the aqueous solutions is kept the same, typically approximately 1 M Na⁺, whereas the annealing temperature can be reduced from 68° C. to 42° C.

The sequencing of the complete cluster of genes responsible for the biosynthesis of thiocoraline (SEQ ID NO: 1) showed the presence of 36 complete open reading frames (ORFs) and of another 2 incomplete ORFs (ORF1 and ORF38, see below). Table 1 (Example 12) shows the position of the different ORFs involved in the biosynthetic thiocoraline production pathway, as well as the amino acid sequences encoded by said ORFs.

The complete chromosomal (genomic) DNA molecule containing the cluster of genes responsible for the biosynthesis of thiocoraline, encoding all the biosynthetic proteins essential for the production of thiocoraline, has been efficiently packaged into two plasmids, specifically into cosmids SuperCos1 and pKC505 (Examples 1 and 2). These two cosmids, containing the cluster of genes responsible for the biosynthesis of thiocoraline, are enough to regenerate the complete biosynthetic pathway for the production of thiocoraline. Therefore, in one particular embodiment, the invention provides the complete cluster of biosynthetic thiocoraline genes in two cosmids which allows having substantially more efficient means for producing thiocoraline.

In one particular embodiment, the nucleic acid molecule of the invention is an optionally purified, isolated nucleic acid molecule comprising a nucleotide sequence encoding a biosynthetic thiocoraline production pathway protein, or a biologically active fragment thereof. In one specific embodiment, the nucleic acid molecule of the invention is selected from the group of genes consisting of:
the nucleic acid molecule comprising nucleotides 2-535 of SEQ ID NO: 1 (orf1);
the nucleic acid molecule comprising nucleotides 993-1130c of SEQ ID NO: 1 (orf2);
the nucleic acid molecule comprising nucleotides 1517-2131 of SEQ ID NO: 1 (tio3);
the nucleic acid molecule comprising nucleotides 2154-2822c of SEQ ID NO: 1 (tio4);
the nucleic acid molecule comprising nucleotides 2970-3791c of SEQ ID NO: 1 (tio5);
the nucleic acid molecule comprising nucleotides 3794-4777c of SEQ ID NO: 1 (tio6);
the nucleic acid molecule comprising nucleotides 4904-5611 of SEQ ID NO: 1 (tio7);
the nucleic acid molecule comprising nucleotides 5701-6426c of SEQ ID NO: 1 (tio8);
the nucleic acid molecule comprising nucleotides 6426-7688c of SEQ ID NO: 1 (tio9);
the nucleic acid molecule comprising nucleotides 7733-8524c of SEQ ID NO: 1 (tio10);
the nucleic acid molecule comprising nucleotides 8791-10002 of SEQ ID NO: 1 (tio11);
the nucleic acid molecule comprising nucleotides 10002-11590c of SEQ ID NO: 1 (tio12);
the nucleic acid molecule comprising nucleotides 11847-13634 of SEQ ID NO: 1 (tio13);
the nucleic acid molecule comprising nucleotides 13734-15005c of SEQ ID NO: 1 (tio14);
the nucleic acid molecule comprising nucleotides 15005-16354c of SEQ ID NO: 1 (tio15);
the nucleic acid molecule comprising nucleotides 16441-18744c of SEQ ID NO: 1 (tio16);
the nucleic acid molecule comprising nucleotides 18774-19055c of SEQ ID NO: 1 (tio17);
the nucleic acid molecule comprising nucleotides 19260-20036 of SEQ ID NO: 1 (tio18);
the nucleic acid molecule comprising nucleotides 20146-20880c of SEQ ID NO: 1 (tio19);
the nucleic acid molecule comprising nucleotides 21188-28969 of SEQ ID NO: 1 (tio20);
the nucleic acid molecule comprising nucleotides 28979-38398 of SEQ ID NO: 1 (tio21);
the nucleic acid molecule comprising nucleotides 38449-38661 of SEQ ID NO: 1 (tio22);
the nucleic acid molecule comprising nucleotides 38642-41263 of SEQ ID NO: 1 (tio23);
the nucleic acid molecule comprising nucleotides 41835-42368 of SEQ ID NO: 1 (tio24);
the nucleic acid molecule comprising nucleotides 42395-43255c of SEQ ID NO: 1 (tio25);
the nucleic acid molecule comprising nucleotides 43340-43741c of SEQ ID NO: 1 (tio26);
the nucleic acid molecule comprising nucleotides 44152-49563 of SEQ ID NO: 1 (tio27);
the nucleic acid molecule comprising nucleotides 49635-53669 of SEQ ID NO: 1 (tio28);
the nucleic acid molecule comprising nucleotides 53749-55305c of SEQ ID NO: 1 (orf29);
the nucleic acid molecule comprising nucleotides 55384-57222c of SEQ ID NO: 1 (orf30);
the nucleic acid molecule comprising nucleotides 57895-58467c of SEQ ID NO: 1 (orf31);
the nucleic acid molecule comprising nucleotides 58535-59206c of SEQ ID NO: 1 (orf32);
the nucleic acid molecule comprising nucleotides 59298-59564c of SEQ ID NO: 1 (orf33);
the nucleic acid molecule comprising nucleotides 59611-60114c of SEQ ID NO: 1 (orf34);
the nucleic acid molecule comprising nucleotides 60202-60888 of SEQ ID NO: 1 (orf35);
the nucleic acid molecule comprising nucleotides 60960-62240 of SEQ ID NO: 1 (orf36);
the nucleic acid molecule comprising nucleotides 62300-62833 of SEQ ID NO: 1 (orf37);
the nucleic acid molecule comprising nucleotides 62925-64650 of SEQ ID NO: 1 (orf38); or
   fragments thereof encoding biologically active fragments of biosynthetic thiocoraline production pathway proteins.

In another particular embodiment, the nucleic acid molecule of the invention is an optionally purified, isolated nucleic acid molecule, comprising a nucleotide sequence encoding two or more biosynthetic thiocoraline production pathway proteins, or biologically active fragments thereof. In one specific embodiment, the nucleic acid molecule of the invention comprises a nucleotide sequence comprising two or more genes selected from the genes identified as orf1, orf2, tio3, tio4, tio5, tio6, tio7, tio8, tio9, tio10, tio11, tio12, tio13, tio14, tio15, tio16, tio17, tio18, tio19, tio20, tio21, tio22, tio23, tio24, tio25, tio26, tio27, tio28, orf29, orf30, orf31, orf32, orf33, orf34, orf35, orf36, orf37, orf38 and fragments thereof encoding biologically active fragments of biosynthetic thiocoraline production pathway proteins.

In another particular embodiment, the nucleic acid molecule of the invention is an optionally purified, isolated nucleic acid molecule, comprising a nucleotide sequence encoding at least one biosynthetic thiocoraline production pathway protein, or a biologically active fragment thereof, or a mutant or variant thereof, wherein said protein is selected from the group consisting of the proteins identified as ORF1 (SEQ ID NO: 2), ORF2 (SEQ ID NO: 3), Tio3 (SEQ ID NO: 4), Tio4 (SEQ ID NO: 5), Tio5 (SEQ ID NO: 6), Tio6 (SEQ ID NO: 7), Tio7 (SEQ ID NO: 8), Tio8 (SEQ ID NO: 9), Tio9 (SEQ ID NO: 10), Tio10 (SEQ ID NO: 11), Tio11 (SEQ ID NO: 12), Tio12 (SEQ ID NO: 13), Tio13 (SEQ ID NO: 14), Tio14 (SEQ ID NO: 15), Tio15 (SEQ ID NO: 16), Tio16 (SEQ ID NO: 17), Tio17 (SEQ ID NO: 18), Tio18 (SEQ ID NO: 19), Tio19 (SEQ ID NO: 20), Tio20 (SEQ ID NO: 21), Tio21 (SEQ ID NO: 22), Tio22 (SEQ ID NO: 23), Tio23 (SEQ ID NO: 24), Tio24 (SEQ ID NO: 25), Tio25 (SEQ ID NO: 26), Tio26 (SEQ ID NO: 27), Tio27 (SEQ ID NO: 28), Tio28 (SEQ ID NO: 29), ORF29 (SEQ ID NO: 30), ORF30 (SEQ ID NO: 31), ORF31 (SEQ ID NO: 32), ORF32 (SEQ ID NO: 33), ORF33 (SEQ ID NO: 34), ORF34 (SEQ ID NO: 35), ORF35 (SEQ ID NO: 36), ORF36 (SEQ ID NO: 37), ORF37 (SEQ ID NO: 38), ORF38 (SEQ ID NO: 39). Said proteins can be obtained from the corresponding aforementioned orfs (orf1, orf2, tio3, tio4, tio5, tio6, tio7, tio8, tio9, tio10, tio11, tio12, tio13, tio14, tio15, tio16, tio17, tio18, tio19, tio20, tio21, tio22, tio23, tio24, tio25, tio26, tio27, tio28, orf29, orf30, orf31, orf32, orf33, orf34, orf35, orf36, orf37, orf38) of the cluster of genes responsible for the biosynthesis of thiocoraline (SEQ ID NO: 1), or from the corresponding regions, mutants or variants thereof.

In another particular embodiment, the nucleic acid molecule of the invention is an optionally purified, isolated nucleic acid molecule comprising a nucleotide sequence encoding at least one variant of a biosynthetic thiocoraline production pathway protein, or a biologically active fragment thereof, wherein said variant is at least 30%, advantageously 50%, preferably 60%, more preferably 70%, even more preferably 80%, particularly 90%, more particularly 95% or more, identical in its amino acid sequence to that of a protein selected from the proteins the amino acid sequences of which are shown in SEQ ID NO: 2-39, or to biologically active fragments thereof. Said variant conserves at least one of the biological activities of functions of the corresponding protein encoded by any of the orfs of the cluster of genes responsible for the biosynthesis of thiocoraline.

In another aspect, the present invention relates to a composition comprising at least one nucleic acid molecule of the invention, preferably an isolated nucleic acid molecule. In one particular embodiment, said composition comprises a nucleic acid molecule of the invention. In another particular embodiment, said composition comprises two or more nucleic acid molecules of the invention. Said nucleic acid molecules can be both of DNA and of RNA.

The nucleic acid molecule of the invention can be isolated from any organism producing thiocoraline either naturally or recombinantly, because the cluster of genes responsible for the biosynthesis of thiocoraline has been inserted in a suitable host cell; nevertheless, in one particular embodiment, said nucleic acid molecule of the invention has been isolated from the marine actinomycete Micromonospora sp. ML1 (see experimental part, Step 1, Examples 1-4).

The isolation and characterization of (chromosomal) genomic DNA and of cloned recombinant DNA from suitable host cells can be carried out by means of conventional or severe hybridization techniques, using the entire or part of a nucleotide sequence as a probe for tracing a suitable gene library.

Therefore, in another aspect, the invention relates to a probe comprising a nucleic acid molecule of the invention or a fragment thereof. In general, the suitably comprise a sequence of at least 5, 10, 15, 20, 25, 30, 40, 50, 60 or more nucleotides. The sequences with a length of 20 to 60 nucleotides are preferred. In one particular embodiment, said probe can be used to detect genes involved in the biosynthesis of thiocoraline in Micromonospora sp. The use of said probe to detect a nucleic acid, e.g., gDNA, cDNA or mRNA, related to the biosynthesis of thiocoraline forms an additional aspect of this invention.

Alternatively, the isolation and characterization of (chromosomal) genomic DNA and of the cloned recombinant DNA from suitable host cells can be carried out by means of techniques based on the enzymatic amplification of nucleic acids. By way of illustration, initiator oligonucleotides can be designed (based on the known sequences of DNA and of proteins involved in the biosynthesis of thiocoraline) which can be used in enzymatic amplification reactions, PCR for example, to amplify and identify other identical or related sequences.

The nucleic acid molecules of the invention can be isolated and, if desired, purified by conventional methods. Although the nucleic acid molecules of the invention will generally be obtained by recombinant or isolation methods, the invention also contemplates the possibility that the nucleic acid molecules of the invention are obtained by chemical synthesis, which molecules will have the same, or substantially the same structure as those derived from both wild-type (wt) and mutant thiocoraline-producing organisms.

In another aspect, the invention relates to a vector, hereinafter vector of the invention, comprising a nucleic acid molecule of the invention encoding at least one biosynthetic thiocoraline production pathway protein, or a biologically active fragment thereof. In one particular embodiment, the vector of the invention is a biologically functional vector or plasmid, such as cloning vector or an expression vector.

In one specific embodiment, the vector of the invention is a cloning vector, preferably a cosmid. Preferred cloning vectors are selected by their capacity to incorporate large DNA sequences (e.g., complete clusters of genes involved in the biosynthesis of products of interest). Said vectors are generally conventional vectors and are commonly available. The present invention further contemplates that the genetic material can be reduced so as to be finally contained in a single cloning vector or plasmid (e.g., cosmid) by means of genetic manipulation by techniques known by persons skilled in the art. The rearrangement can be carried out by means of cloning, PCR or synthetic genes or combination of any of these techniques known in the state of the art.

In another particular embodiment, the vector of the invention is an expression vector suitable for its insertion into a suitable host cell. The insertion of said vector into said suitable host cell can be carried out by any conventional genetic material transfer method (e.g., transformation, transfection, etc.).

Therefore, in another aspect, the invention relates to a host cell, hereinafter host cell of the invention, transformed or transfected with a vector of the invention. Said host cell of the invention contains one or more nucleic acid molecules of the invention. In one particular embodiment, the host cell of the invention contains a nucleic acid molecule of the invention. In another particular embodiment, the host cell of the invention contains two or more nucleic acid molecules of the invention; in this case, said nucleic acid molecules of the invention can be identical of different from one another.

A preferred host cell of the invention is a host cell stably transformed or transfected with a vector of the invention comprising an (exogenous) nucleic acid molecule of the invention comprising a nucleotide sequence encoding at least one biosynthetic thiocoraline production pathway protein, or a biologically active fragment thereof, in a manner sufficient to direct the biosynthesis and/or rearrangement of thiocoraline. The host cell is preferably a microorganism, more preferably a bacterium. In one particular embodiment, said host cell is a Gram-positive bacterium, such as an actinomycete, a streptomycete for example.

Although different streptomycete species such as Streptomyces coelicolor, Streptomyces lividans, Streptomyces albus and Streptomyces avermitilis have been used in the examples of the present invention as heterologous hosts, the heterologous expression of the genes involved in the biosynthesis of thiocoraline can be carried out in other streptomycetes, actinomycetes, etc., provided that they can be transformed, preferably in a stable manner, with the vectors of the invention. The in vitro expression of the proteins can be carried out, if desired, using conventional methods.

In one particular embodiment, the invention provides a host cell of the invention, such as a recombinant bacterium for example, in which at least one region of the nucleic acid molecule of the invention has been altered to give rise to a recombinant host cell, such as a recombinant bacterium, producing altered thiocoraline levels compared to the corresponding non-recombinant, i.e. wt, thiocoraline-producing cell (bacterium). To that end, conventional techniques known by persons skilled in the art can be used, which include for example increasing the number of copies of the genes responsible for the most important domains of the NRPSs involved in the production of thiocoraline or increasing the gene expression-regulating sequences of those genes by genetic engineering techniques known in the state of the art and thus increasing the yield in the production of thiocoraline.

In another aspect, the invention relates to a protein, hereinafter protein of the invention, encoded by the nucleic acid molecule of the invention.

As used herein, the term “protein” means polypeptides, enzymes and the like, encoded by the nucleic acid molecule of the invention comprised by the biosynthetic pathway for the production of thiocoraline. The proteins of the invention include amino acid chains with variable lengths, including full-length amino acid chains, wherein the amino acid moieties are joined by covalent peptide bonds, as well as biologically active fragments of said proteins involved in the biosynthesis of thiocoraline, as well as the biologically active variants thereof. The proteins of the invention can be natural, recombinant or synthetic. By way of illustration, said proteins involved in the biosynthesis of thiocoraline can be produced through conventional recombinant DNA technology, inserting a nucleotide sequence encoding the protein into a suitable expression vector and expressing the protein in a suitable host cell or through conventional chemical peptide synthesis, for example, by means of the solid-phase peptide synthesis of Merrifield (Merrifield, J. Am. Chem. Soc. 85:2149-2154 (1963)) in which the amino acids are individually and sequentially joined to the amino acid chain. Alternatively, the proteins of the invention can be synthesized using equipment for automated protein synthesis marketed by different manufacturers (e.g., Perkin-Elmer, Inc.).

The biologically active variants included within the scope of the present invention comprise at least one biologically active fragment of the amino acid sequence encoded by the nucleic acid molecule of the invention, i.e., a part of the protein structure retaining the active function of the protein, for example, the thioesterase part encoded by the tio18 gene having the same or substantially the same activity as the Tio18 protein encoded by said tio18 gene, i.e., it has at least a similarity or power of at least approximately 70%, advantageously of at least 80%, preferably of at least 90%, more preferably of about 95% approximately.

The biologically active variants of the proteins of the invention include active amino acid structures in which amino acids, naturally occurring alleles, etc. have been eliminated, substituted or added. The biologically active fragment can be easily identified by subjecting the full-length protein to chemical or enzymatic digestion in order to prepare fragments and then assaying the amino acid structure fragments conserving the same or substantially the same biological activity as the full-length protein.

In one particular embodiment, the protein of the invention is an optionally purified, isolated protein involved in the biosynthesis of thiocoraline encoded by a gene selected from the group consisting of the genes identified as orf1, orf2, tio3, tio4, tio5, tio6, tio7, tio8, tio9, tio10, tio11, tio12, tio13, tio14, tio15, tio16, tio17, tio18, tio19, tio20, tio21, tio22, tio23, tio24, tio25, tio26, tio27, tio28, orf29, orf30, orf31, orf32, orf33, orf34, orf35, orf36, orf37 and orf38.

In another particular embodiment, the protein of the invention is an optionally purified, isolated protein involved in the biosynthesis of thiocoraline selected from the group consisting of the proteins identified as ORF1 (SEQ ID NO: 2), ORF2 (SEQ ID NO: 3), Tio3 (SEQ ID NO: 4), Tio4 (SEQ ID NO: 5), Tio5 (SEQ ID NO: 6), Tio6 (SEQ ID NO: 7), Tio7 (SEQ ID NO: 8), Tio8 (SEQ ID NO: 9), Tio9 (SEQ ID NO: 10), Tio10 (SEQ ID NO: 11), Tio11 (SEQ ID NO: 12), Tio12 (SEQ ID NO: 13), Tio13 (SEQ ID NO: 14), Tio14 (SEQ ID NO: 15), Tio15 (SEQ ID NO: 16), Tio16 (SEQ ID NO: 17), Tio17 (SEQ ID NO: 18), Tio18 (SEQ ID NO: 19), Tio19 (SEQ ID NO: 20), Tio20 (SEQ ID NO: 21), Tio21 (SEQ ID NO: 22), Tio22 (SEQ ID NO: 23), Tio23 (SEQ ID NO: 24), Tio24 (SEQ ID NO: 25), Tio25 (SEQ ID NO: 26), Tio26 (SEQ ID NO: 27), Tio27 (SEQ ID NO: 28), Tio28 (SEQ ID NO: 29), ORF29 (SEQ ID NO: 30), ORF30 (SEQ ID NO: 31), ORF31 (SEQ ID NO: 32), ORF32 (SEQ ID NO: 33), ORF33 (SEQ ID NO: 34), ORF34 (SEQ ID NO: 35), ORF35 (SEQ ID NO: 36), ORF36 (SEQ ID NO: 37), ORF37 (SEQ ID NO: 38), ORF38 (SEQ ID NO: 39), and combinations thereof, or biologically active fragments thereof. The hypothetical functions of said proteins are included in Table 1.

The orfs of the cluster of genes responsible for the biosynthesis of thiocoraline, encoding the proteins involved in the biosynthesis of said compound, can be identified using conventional techniques. Illustrative non-limiting examples of said techniques include computational analysis for locating the stop and start codons, the putative locations of the reading frames based on the frequencies of the codons, alignments by similarity to genes expressed in other actinomycetes and the like. The proteins of the invention can thus be identified using the nucleotide sequence of the present invention and the orfs or the proteins encoded by them can be isolated and if desired, purified, or alternatively, synthesized by chemical methods. Gene constructs for the expression of said products based on the orfs can be designed and the suitable expression regulating elements (promoters, terminators, etc.) can be included and said gene constructs can be introduced in suitable host cells for expressing the protein or proteins encoded by one or more orfs.

The proteins of the invention can be isolated and, if desired, purified by conventional methods. The proteins are preferably obtained in a substantially pure form, although a lower degree of purity, typically from 80% to 90% approximately, can also be acceptable. The invention also contemplates the possibility that the proteins of the invention are obtained by chemical synthesis, which proteins will have the same or substantially the same structure as those directly derived from both wild-type (wt) and mutant thiocoraline-producing organisms.

In another aspect, the invention relates to a process for producing a protein of the invention involved in the biosynthesis of thiocoraline which comprises growing, under suitable (nutrient and environmental) conditions, a thiocoraline-producing organism and, if desired, isolating one or more of said proteins involved in the biosynthesis of thiocoraline. If desired, said protein of the invention can be isolated and purified by conventional methods, such as those described previously.

In another aspect, the invention relates to a method for producing thiocoraline which comprises growing, under suitable conditions for producing said compound, a thiocoraline-producing organism in which the number of copies of genes encoding proteins involved in the biosynthesis of thiocoraline has been increased, and, if desired, isolating thiocoraline.

In one particular embodiment, the thiocoraline-producing organism is an actinomycete such as Micromonospora sp for example, in which the number of copies of genes encoding proteins involved in the biosynthesis of thiocoraline has been increased. The increase in the number of copies of genes encoding proteins involved in the biosynthesis of thiocoraline can be carried out by conventional methods known by persons skilled in the art. In this case, the previously described method comprises fermenting said organism under suitable nutrient and environmental conditions for the expression of the genes involved in the production of thiocoraline. If desired, the thiocoraline produced can be isolated and purified from the culture medium by conventional methods.

In another aspect, the invention relates to a method for producing thiocoraline which comprises growing, under suitable conditions for producing said compound, a thiocoraline-producing organism in which the expression of the genes encoding the proteins responsible for the biosynthesis of thiocoraline has been modulated by means of manipulating or substituting one or more genes encoding proteins involved in the biosynthesis of thiocoraline or by means of manipulating the sequences responsible for regulating the expression of said genes, and, if desired, isolating thiocoraline. The expression of the genes encoding said proteins responsible for the biosynthesis of thiocoraline has preferably been improved. To that end, the unessential gene sequences in the thiocoraline biosynthesis process can be eliminated, or the efficiency of the gene expression-regulating sequences of said genes can be increased by genetic engineering sequences known by persons skilled in the art. The yield in the production of thiocoraline can thus be increased. The genetic manipulation for eliminating the unessential gene sequences in the thiocoraline biosynthesis process or for increasing the efficiency of the gene expression-regulating sequences of said genes can be carried out by genetic engineering techniques known by persons skilled in the art.

In one particular embodiment, the thiocoraline-producing organism is an actinomycete such as Micromonospora sp for example, in which the expression of the genes encoding the proteins responsible for the biosynthesis of thiocoraline has been modulated by means of manipulating or substituting one or more genes encoding proteins involved in the biosynthesis of thiocoraline or by means of manipulating the sequences responsible for regulating the expression of said genes, which can be carried out by conventional methods known by persons skilled in the art. In this case, the previously described method comprises fermenting said organism under suitable nutrient and environmental conditions for the expression of the genes involved in the production of thiocoraline. If desired, the thiocoraline produced can be isolated and purified from the culture medium by conventional methods.

In another aspect, the invention relates to a method for producing thiocoraline which comprises growing, under suitable conditions for producing said compound, a host cell of the invention transformed or transfected with a vector of the invention comprising the cluster of genes responsible for the biosynthesis of thiocoraline, and, if desired, isolating thiocoraline. The (nutrient, environmental, etc.) conditions will be selected according to the nature of the host cell.

In one particular embodiment, the host cell of the invention is selected from an organism producing thiocoraline natively, an organism that does not produce thiocoraline natively and an organism that has been genetically manipulated to produce thiocoraline. In one particular embodiment, said host cell of the invention is an actinomycete or a streptomycete.

In another aspect, the invention relates to a process, based on the use of genes responsible for the biosynthesis of thiocoraline from Micromonospora sp. ML1, for the production of said compound in another actinomycete, which comprises:
(1) obtaining mutants affected in specific genes of the thiocoraline biosynthesis pathway;
(2) isolating the Micromonospora sp. ML1 chromosome region containing the cluster of genes responsible for the biosynthesis of thiocoraline;
(3) obtaining and analyzing the nucleotide sequence of the cluster of genes responsible for the biosynthesis of thiocoraline; and
(4) heterologously producing thiocoraline in other actinomycetes.

The identification and isolation of the Micromonospora sp. ML1 chromosome region containing the cluster of genes responsible for the biosynthesis of thiocoraline, as well as the analysis of the nucleotide sequence of said cluster can be carried out based on the teachings provided by this invention, illustrated in a non-limiting manner in the Examples attached to this description.

The mutants affected in specific genes of the thiocoraline biosynthesis pathway can be identified by conventional methods. In one particular embodiment, said mutants can be identified by means of culturing and measuring the production of thiocoraline by conventional methods, by HPLC-MS for example, as mentioned in Example 5.

The entire or part of the cluster of genes responsible for the biosynthesis of thiocoraline can be introduced in an actinomycete by conventional methods, e.g., by transformation or transfection, for the heterologous production of thiocoraline by fermenting a suitable nutrient medium under the suitable conditions for the production of thiocoraline and, if desired, the thiocoraline thus obtained can be isolated and/or purified by conventional methods.

The determination of the cluster of genes responsible for the biosynthesis of thiocoraline has a great commercial importance. The isolation and complete description of the cluster of genes responsible for the biosynthesis of thiocoraline provided by this invention allows increasing the production of thiocoraline and manipulating thiocoraline-producing organisms. In this sense, the number of copies of the genes responsible for the most important domains of the NRPSs involved in the production of thiocoraline can be increased or the efficiency of the gene expression-regulating sequences of those genes can be increased by genetic engineering techniques known in the state of the art and the yield in its production can thus be increased.

Another advantage associated to the identification and cloning of the complete cluster of thiocoraline genes provided by the present invention relates to the efficient production of thiocoraline. In fact, it allows obtaining a compound of great interest in a smaller number of steps. The elimination of unessential sequences in the biosynthesis process in cluster mutants considerably reduces the time necessary for producing the compound of interest. The remaining sequences are sufficient and maintain their functionality for producing thiocoraline.

### EXPERIMENTAL PART

The experimental procedures of the present invention include conventional molecular biology methods in the current state of the art. Detailed descriptions of the techniques that are not explained herein can be found in the manuals of Kieser et al. (Practical Streptomyces genetics. The John Innes Foundation, Norwich, Great Britain, 2000) and Sambrook et al. (Molecular cloning: a laboratory manual. Cold Spring Harbor Laboratory Press, Cold Spring Harbor, N.Y., USA, 2001). The following steps describe in detail the present invention without limitation.

Step 1. Isolating the Micromonospora sp. ML1 Chromosome Region Containing the Thiocoraline Biosynthesis Pathway Genes

### Example 1

### Construction of a Gene Library in SuperCos1 from Micromonospora sp. ML1 Chromosomal DNA

Chromosomal DNA was obtained using the salting out protocol (Kieser et al. 2000) from a Micromonospora sp. ML1 culture (Espliego, F. Ph.D. Thesis, 1996, University of Leon; de la Calle, F. Ph.D. Thesis, 1998, Autonomous University of Madrid), available in the Pharma Mar, S.A. culture collection, in MIAM2 medium (5 g/l of yeast extract, 3 g/l of meat extract, 5 g/l of tryptone, 5 g/l of glucose, 20 g/l of dextrin, 4 g/l of CaCO₃, 10 g/l of sea salts. pH 6.8). This chromosomal DNA was subjected to partial digestion with the BamHI endonuclease and the fragments obtained were used to generate a gene library in the cosmid SuperCos 1 (Stratagene), digested with BamHI. The generation of this gene library in E. coli XL-1 Blue MR (Stratagene) was carried out according to already described procedures (Sambrook et al. 2001) and the in vitro packaging kit Gigapack III Gold Packaging Extract Kit (Stratagene).

1,000 E. coli transducing colonies were deposited on nylon membranes in order to conduct an in situ colony hybridization analysis by means of usual protocols (Sambrook et al. 2001).

### Example 2

### Construction of a Gene Library in pKC505 from Micromonospora sp. ML1 Chromosomal DNA

Chromosomal DNA was obtained using the salting out protocol (Kieser et al. 2000) from a Micromonospora sp. ML1 culture in MIAM2 medium. This chromosomal DNA was subjected to partial digestion with the Sau3AI endonuclease and the fragments obtained were used to generate a gene library in the bifunctional cosmid Escherichia coli/Streptomyces pKC505 (Richardson at al. 1987, Gene 61, 231-241), digested with BamHI. The generation of this gene library in E. coli ED8767 was carried out according to already described procedures (Sambrook et al. 2001) and the in vitro packaging kit Gigapack III Gold Packaging Extract Kit (Stratagene).

3,300 E. coli transducing colonies were deposited on 96-well microtiter plates containing TSB medium (Merck) with 25 μg/ml of apramycin and incubated at 30° C. for 24 hours. These clones were replicated to TSA (Tryptic Soy Agar) plates with 25 μg/ml of apramycin, and after one night at 30° C., the colonies were transferred to nylon membranes in order to conduct an in situ colony hybridization analysis by means of usual protocols (Sambrook et al. 2001).

### Example 3

### Design of Specific Oligonucleotides for Adenylation Domains in NRPS and their PCR Amplification from Micromonospora sp. ML1 Chromosomal DNA

Based on the structure of thiocoraline, the NRPSs responsible for its biosynthesis were expected to have from one to three adenylation domains activating L-cysteine and one domain activating glycine. On this basis, degenerated oligonucleotides, based on conserved regions inside the NRPS adenylation domains, which can specifically amplify DNA fragments encoding NRPS adenylation domains which were combined with oligonucleotides described in the literature for the amplification of NRPS adenylation domains were designed.

The PCR amplification with the initiator oligonucleotides:

MTF2 (5′-GCNGGYGGYGCNTAYGTNCC-3′) (SEQ ID NO:40); Neilan et al. 1999. J. Bacteriol. 181(13):4089-4097) and

PSV-4 (5-SAGSAGGSWGTGGCCGCCSAGCTCGAAGAA-3′) (SEQ ID NO:41) resulted in a 1.3 kb band which was cloned into a pGEM-T Easy vector (Promega). The PCR program used was an initial cycle of 95° C.-2 min; 60° C.-15 min; 72° C.-6 min followed by 20 cycles of 95° C.-1 min; 60° C.-2 min; 72° C.-2 min. Micromonospora sp. ML1 chromosomal DNA was used as a template.

The analysis of the clones by restriction fragment length polymorphism (RFLP) showed that there were three types of different clones corresponding to peptide synthetases, pGPSV1, pGPSV2 and pGPSV3, which contained the adenylation domains fragments called PSV1, PSV2 and PSV3, respectively.

The insert of the clones was subsequently released with an EcoRI digestion and the fragment was cloned into pBBR1-MCS2 (Kovach, M. E. et al. 1995. Gene. 166:175-176) to construct plasmids pBPSV1, pBPSV2 and pBPSV3, respectively, which contained the adenylation domains fragments called PSV1, PSV2 and PSV3, respectively.

From the PCR band obtained with initiator oligonucleotides MTF2 and PS4, a nested-PCR (30 cycles of 95° C.-1 min; 60° C.-1 min; 72° C.-1 min) was carried out with the initiator oligonucleotides PS2-TG: 5′-ACNGGNMRNCCNAARGG-3′ (SEQ ID NO:42) and MTR: 5′-CCNCGDATYTTNACY-3 (SEQ ID NO:43) (Neilan et al. 1999. J. Bacteriol. 181(13):4089-4097) in order to obtain a 750 bp band which was cloned into a pGEM-T Easy vector (Promega). The analysis of the clones by RFLP showed that there were two new types of different clones corresponding to peptide synthetases, pGPSV4 and pGPSV5 respectively, which contained the adenylation domain fragments called PSV4 and PSV5, respectively.

The PCR amplification with the initiator oligonucleotides PS2M: 5′-TACACSGGCWSSACSGG-3′ (SEQ ID NO:44) and PSV-4 resulted in a 1.3 kb band which was cloned into a pGEM-T Easy vector (Promega). The program used was a Touch-down starting with 5 cycles at the annealing temperature of 72° C., followed by 10 cycles at 70° C. of annealing to end with 20 cycles at 68° C. (96° C.-1 min; 72° C.-68° C.-2 min; 72° C.-3 min). The analysis of the clones by RFLP showed that was a new type of clone corresponding to a peptide synthetase, pGPSV6, which contained the adenylation domain fragment called PSV6.

### Example 4

### Analysis of the Gene Libraries by Colony Hybridization

The gene libraries constructed in SuperCos1 and in pKC505 (Examples 1 and 2) were subjected to respective in situ colony hybridization analyses (Sambrook et al. 2001) using the DIG DNA Labeling and Detection Kit system (Roche). The 6 adenylation domain fragments called PSV1-PSV6 were used as probes.

The following was obtained from the gene library constructed in SuperCos1:
3 positive cosmids (clones) which hybridized with fragment PSV1, called pCT1a, pCT1b and pCT1c;
3 positive cosmids (clones) which hybridized with fragment PSV2, called pCT2a, pCT2b and pCT2c; from these fragments, pCT2c also hybridized with fragment PSV5;
2 positive cosmids (clones) which hybridized with fragment PSV3, called pCT3a and pCT3b; furthermore, both of them also hybridized with fragment PSV6; and
1 positive cosmid (clone) which hybridized with PSV4, called pCT4a.

55 positive cosmids were obtained from the gene library constructed in pKC505:
10 positive cosmids (clones) which hybridized with fragment PSV2, called cosV1-F8, cosV7-D2, cosV7-D12, cosV14-H4, cosV19-B4, cosV29-B9, cosV31-B11, cosV31-H10, cosV33-D12, cosV33-F7;
7 positive cosmids which hybridized with fragment PSV5, called cosV1-B6, cosV6-H8, cosV11-F10, cosV20-F8, cosV22-F7, cosV25-B3, cosV32-B4; and
38 positive cosmids which hybridized with fragments PSV1, PSV3, PSV4 or PSV6, called cosV1-B7, cosV1-F5, cosV2-E5, cosV2-F11, cosV3-D9, cosV4-D2, cosV5-D7, cosV5-G6, cosV6-A7, cosV6-A12, cosV7-E7, cosV8-F8, cosV9-H7, cosV10-A3, cosV11-B4, cosV11-G2, cosV12-B12, cosV13-B2, cosV16-H11, cosV17-A3, cosV19-F4, cosV20-B3, cosV20-H5, cosV2′-H6, cosV22-B11, cosV23-F8, cosV26-H11, cosV28-G1, cosV29-E1, cosV29-G6, cosV30-G5, cosV3′-A12, cosV3′-E10, cosV32-A7, cosV32-D10, cosV33-A8, cosV33-D10, cosV33-F10,

### Step 2. Generating Mutants in Six Isolated Adenylation Regions

The six adenylation domain fragments previously amplified from Micromonospora sp. ML1 chromosomal DNA (PSV1, PSV2, PSV3, PSV4, PSV5 and PSV6) were used for independent gene interruption experiments for the purpose of evaluating the regions involved in the biosynthesis of thiocoraline (Examples 6-11).

The conjugative plasmid E. coli/Streptomyces pOJ260 (Bierman et al. 1992, Gene 116, 43-49) was used to generate constructs pFL903, pFL904, pFL905, pFL906, pFL940 and pFL941 which contained regions PSV1 to PSV6, respectively. These constructs were introduced in the conjugative E. coli ET12567 (pUB307) strain (Kieser et al. 2000) and from here, by conjugation, in the Micromonospora sp. ML1 strain, using described procedures (Kieser et al. 2000). The transconjugant clones were selected with apramycin and the integration in the suitable chromosomal region was verified by means of Southern hybridization using the corresponding regions of the adenylation domain fragments PSV1 to PSV6. The transconjugants selected from each region of the PSV adenylation domains (PSV1-PSV6) were grown in thiocoraline production medium MT4 and their mycelium was subsequently extracted with acetonitrile and analyzed by HPLC-MS (Example 5). Only the mutants affected in the adenylation domains PSV2 and PSV5 has a phenotype that does not produce thiocoraline (Examples 7 and 10). The production of thiocoraline in mutants with deletions in PSV1, PSV3, PSV4 and PSV6 was similar to that of the wt strain (Examples 6, 8, 9 and 11). These experiments showed that the adenylation domains PSV2 and PSV5 were involved in the biosynthesis of thiocoraline.

### Example 5

### HPLC Detection of the Production of Thiocoraline

The extracts with acetonitrile of the different analyzed strains were concentrated in the rotary evaporator and resuspended in DMSO before being used in HPLC-MS analysis.

The samples (10 μl) were analyzed by HPLC, using a reversed-phase column (Symmetry C₁₈, 2.1×150 mm, Waters), using acetonitrile and a mixture of 0.1% of trifluoroacetic acid in water as solvents. During the first 4 minutes, a concentration of the mobile phase with 10% of acetonitrile was maintained isocratically. Then, up to 30 minutes, a linear gradient from 10% to 100% of acetonitrile is started. The flow used was 0.25 ml/min. The spectral detection and characterization of the peaks was carried out using a photodiode detector and by means of using the Millennium computer software (Waters). The chromatograms were extracted at an absorbance of 230 nm.

### Example 6

### Gene Interruption in the PSV1 Region

The PSV1 region was obtained from plasmid pBPSV1 as an 1.3 kb EcoRI band and was cloned into the EcoRI site of conjugative plasmid E. coli/Streptomyces pOJ260, generating pFL903. pOJ260 contains a gene conferring apramycin resistance in Streptomyces and in these cells it is a suicide plasmid.

The construct pFL903 was introduced in the conjugative E. coli ET12567 (pUB307) strain and from there, by conjugation, in the Micromonospora sp. ML1 strain, using described procedures (Kieser et al. 2000). The transconjugant clones were selected with 25 μg/ml of apramycin and, from the chromosomal DNA thereof, it was verified that the PSV1 region has indeed been interrupted by means of Southern hybridization. The probe used in this case was the PSV1 band.

The mutant Micromonospora sp. ΔPSV1 was grown in thiocoraline production medium MT4 and its mycelium was subsequently extracted with acetonitrile and analyzed by HPLC-MS (see Example 5), proving to be a thiocoraline producer. The composition of the culture medium MT4 per liter is as follows: 6 g soy flour, 2.5 g of malt extract, 2.5 g of peptone, 5 g of dextrose, 20 g of dextrin, 4 g of CaCO₃, 10 g of sea salts, adjust the pH to 6.8.

### Example 7

### Gene Interruption in the PSV2 Region

The PSV2 region was obtained from plasmid pBPSV2 as a 1.3 kb EcoRI band and was cloned into the EcoRI site of plasmid pOJ260, generating pFL904.

The construct pFL904 was introduced in the conjugative E. coli ET12567 strain (pUB307) and from there, by conjugation, in the Micromonospora sp. ML1 strain. The transconjugant clones were selected with 25 μg/ml of apramycin and, from the chromosomal DNA thereof, it was verified that the PSV2 region has indeed been interrupted by means of Southern hybridization. The probe used in this case was the PSV2 band.

The mutant Micromonospora sp. ΔPSV2 was grown in thiocoraline production medium MT4 and its mycelium was subsequently extracted with acetonitrile and analyzed by HPLC-MS (Example 5), giving as a result that this strain did not produce thiocoraline.

### Example 8

### Gene Interruption in the PSV3 Region

The PSV3 region was obtained from plasmid pBPSV3 as a 1.4 kb EcoRI band and was cloned into the EcoRI site of plasmid pOJ260, generating pFL905.

The construct pFL905 was introduced in the conjugative E. coli ET12567 (pUB307) strain and from there, by conjugation, in the Micromonospora sp. ML1 strain. The transconjugant clones were selected with 25 μg/ml of apramycin and, from the chromosomal DNA thereof, it was verified that the PSV3 region had indeed been interrupted by means of Southern hybridization. The probe used in this case was the PSV3 band.

The mutant Micromonospora sp. ΔPSV3 was grown in thiocoraline production medium MT4 and its mycelium was subsequently extracted with acetonitrile and analyzed by HPLC-MS (Example 5), proving to be a thiocoraline producer.

### Example 9

### Gene Interruption in the PSV4 Region

The PSV4 region was obtained from plasmid pGPSV4 as a 1.2 kb EcoRI band and was cloned into the EcoRI site of plasmid pOJ260, generating pFL906.

The construct pFL906 was introduced in the conjugative E. coli ET12567 (pUB307) strain and from there, by conjugation, in the Micromonospora sp. ML1 strain. The transconjugant clones were selected with 25 μg/ml of apramycin and from the chromosomal DNA thereof, it was verified that the PSV4 region had indeed been interrupted by means of Southern hybridization. The probe used in this case was the PSV4 band.

The mutant Micromonospora sp. ΔPSV4 was grown in thiocoraline production medium MT4 and its mycelium was subsequently extracted with acetonitrile and analyzed by HPLC-MS (Example 5), proving to be a thiocoraline producer.

### Example 10

### Gene Interruption in the PSV5 Region

The PSV5 region was obtained from plasmid pGPSV5 as a 1.1 kb EcoRI band and was cloned into the EcoRI site of plasmid pOJ260, generating pFL940.

The construct pFL940 was introduced in conjugative E. coli ET12567 (pUB307) strain and from there, by conjugation, in the Micromonospora sp. ML1 strain. The transconjugant clones were selected with 25 μg/ml of apramycin and, from the chromosomal DNA thereof, it was verified that the PSV5 region had indeed been interrupted by means of Southern hybridization. The probe used in this case was the PSV5 band.

The mutant Micromonospora sp. ΔPSV5 was grown in thiocoraline production medium MT4 and its mycelium was subsequently extracted with acetonitrile and analyzed by HPLC, giving as a result that this strain did not produce thiocoraline.

### Example 11

### Gene Interruption in the PSV6 Region

The PSV6 region was obtained from plasmid pGPSV6 as a 1.1 kb EcoRI band and was cloned into the EcoRI site of plasmid pOJ260, generating pFL941.

The construct pFL941 was introduced in the conjugative E. coli ET12567 (pUB307) strain and from there, by conjugation, in the Micromonospora sp. ML1 strain. The transconjugant clones were selected with 25 μg/ml of apramycin and, from the chromosomal DNA thereof, it was verified that the PSV6 region had indeed been interrupted by means of Southern hybridization. The probe used in this case was the PSV6 band.

The mutant Micromonospora sp. ΔPSV6 was grown in thiocoraline production medium MT4 and its mycelium was subsequently extracted with acetonitrile and analyzed by HPLC, proving to be a thiocoraline producer.

### Step 3. Obtaining and Analyzing the Nucleotide Sequence of the Gene Cluster Responsible for the Biosynthesis of Thiocoraline

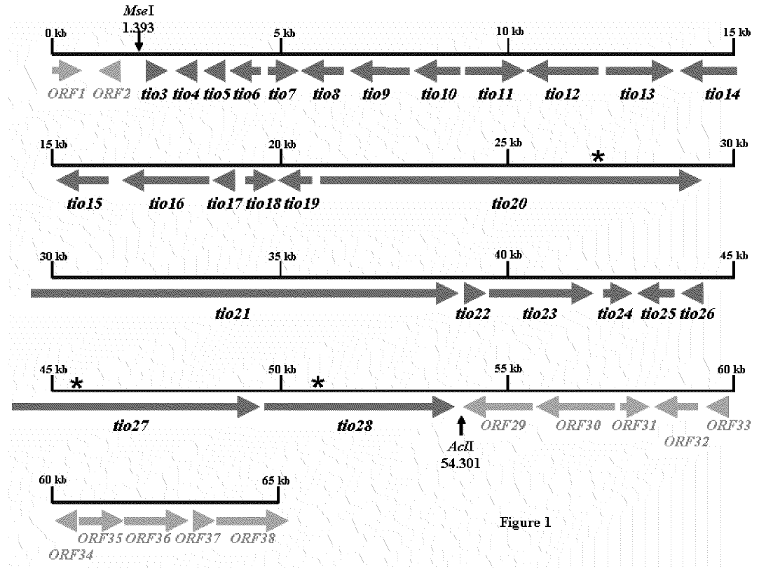

Based on the previous results, in which the amplified areas of the adenylation domains PSV2 and PSV5 were the only ones the gene interruption of which caused a phenotype that did not produce thiocoraline, two overlapping cosmids, cosV33-D12 (containing the region of adenylation domain PSV2) and pCT2c (containing the regions of adenylation domains PSV2 and PSV5), were chosen to be sequenced. The analysis of the 64,650 bp obtained from said cosmids showed the presence of 36 complete ORFs and 2 incomplete ORFs, the organization of which is shown in FIG. 1. The comparison with the protein sequences existing in the databases of the products deduced from the different genes allowed deducing the functions for most of them (Table 1).

### Example 12

### Determination and Analysis of the Nucleotide Sequence of the Insert of Cosmids cosV33-D12 and pCT2c

Both cosmids were sequenced using the usual methodology and the program package GCG, from the Genetics Computer Group of the University of Wisconsin, was used for the computer analysis of the sequence (Devereux et al. 1984, Nucleic Acid Res. 12, 387-395).

A sequence of 64,650 nucleotides was thus obtained, the computer analysis of which showed the existence of 38 ORFs [36 complete ORFs and 2 incomplete ORFs], the organization of which is in FIG. 1. The gene expression products of said ORFs were compared with proteins having a known function present in the databases using the BLAST program (Altschul et al. 1997, Nucleic Acid Res. 25, 3389-3402), whereby the probable functions for most of these ORFs were assigned (Table 1).

TABLE 1

| | | Amino | | |
|---|---|---|---|---|
| Gene | Position | acids | Deduced Function | Notes |
| ORF1 | 2-535 | 178* | Transposase | SEQ ID |
| | | | | NO: 2 |
| ORF2 | 993- | 46 | Unknown | SEQ ID |
| | 1130c | | | NO: 3 |
| Tio3 | 1517- | 205 | OmpR family regulator | SEQ ID |
| | 2131 | | | NO: 4 |
| Tio4 | 2154- | 223 | Possible regulator | SEQ ID |
| | 2822c | | | NO: 5 |
| Tio5 | 2970- | 274 | ABC transporter (permease | SEQ ID |
| | 3791c | | subunit) | NO: 6 |
| Tio6 | 3794- | 328 | ABC transporter (ATPase | SEQ ID |
| | 4777c | | subunit) | NO: 7 |
| Tio7 | 4904- | 236 | MerR family regulator | SEQ ID |
| | 5611 | | | NO: 8 |
| Tio8 | 5701- | 242 | Tryptophan 2,3- | SEQ ID |
| | 6426c | | dioxygenase | NO: 9 |
| Tio9 | 6426- | 421 | Kynurenine | SEQ ID |
| | 7688c | | aminotransferase | NO: 10 |
| Tio10 | 7733- | 264 | NAD- or NADP- | SEQ ID |
| | 8524c | | oxidoreductase | NO: 11 |
| Tio11 | 8791- | 404 | Quinaldate 3-hydroxylase | SEQ ID |
| | 10002 | | (Cytochrome P₄₅₀) | NO: 12 |
| Tio12 | 10022- | 523 | 3-hydroxy-quinaldate-AMP- | SEQ ID |
| | 11590c | | Ligase | NO: 13 |
| Tio13 | 11847- | 596 | NRPS | SEQ ID |
| | 13634 | | | NO: 14 |
| Tio14 | 13734- | 424 | Unknown | SEQ ID |
| | 15005c | | | NO: 15 |
| Tio15 | 15005- | 450 | V-chloroperoxidase | SEQ ID |
| | 16354c | | | NO: 16 |
| Tio16 | 16441- | 768 | NRPS | SEQ ID |
| | 18744c | | | NO: 17 |
| Tio17 | 18774- | 94 | 3-hydroxy-quinaldate- | SEQ ID |
| | 19055c | | Carrier Protein | NO: 18 |
| Tio18 | 19260- | 259 | Thioesterase | SEQ ID |
| | 20036 | | | NO: 19 |
| Tio19 | 20146- | 245 | Thioesterase | SEQ ID |
| | 20880c | | | NO: 20 |
| Tio20 | 21188- | 2594 | NRPS | SEQ ID |
| | 28969 | | | NO: 21 |
| Tio21 | 28979- | 3140 | NRPS | SEQ ID |
| | 38398 | | | NO: 22 |
| Tio22 | 38449- | 71 | Unknown (similar to MbtH) | SEQ ID |
| | 38661 | | | NO: 23 |
| Tio23 | 38642- | 874 | DNA excisionase | SEQ ID |
| | 41263 | | | NO: 24 |
| Tio24 | 41835- | 178 | OmpR family regulator | SEQ ID |
| | 42368 | | | NO: 25 |
| Tio25 | 42395- | 287 | Possible regulator | SEQ ID |
| | 43255c | | | NO: 26 |
| Tio26 | 43340- | 134 | Unknown | SEQ ID |
| | 43741c | | | NO: 27 |
| Tio27 | 44152- | 1804 | NRPS | SEQ ID |
| | 49563 | | | NO: 28 |
| Tio28 | 49635- | 1345 | NRPS | SEQ ID |
| | 53669 | | | NO: 29 |
| ORF29 | 53749- | 519 | Glucoside permease | SEQ ID |
| | 55305c | | | NO: 30 |
| ORF30 | 55384- | 613 | Glucoside permease | SEQ ID |
| | 57222c | | | NO: 31 |
| ORF31 | 57895- | 191 | MarR family regulator | SEQ ID |
| | 58467 | | | NO: 32 |
| ORF32 | 58535- | 224 | Anti anti-σ factor | SEQ ID |
| | 59206c | | | NO: 33 |
| ORF33 | 59298- | 89 | Unknown | SEQ ID |
| | 59564c | | | NO: 34 |
| ORF34 | 59611- | 168 | Anti anti-σ factor | SEQ ID |
| | 60114c | | | NO: 35 |
| ORF35 | 60202- | 229 | Regulator system of two | SEQ ID |
| | 60888 | | components (Response | NO: 36 |
| | | | regulator) | |
| ORF36 | 60960- | 427 | Regulator system of two | SEQ ID |
| | 62240 | | components (Histidine | NO: 37 |
| | | | kinase) | |
| ORF37 | 62300- | 178 | Unknown | SEQ ID |
| | 62833 | | | NO: 38 |
| ORF38 | 62925- | 574* | Chaperon DnaK | SEQ ID |
| | 64650 | | | NO: 39 |

*Incomplete ORF

Some of the identified proteins are involved in the formation of the thiocoraline peptide structure, such as for example several of the identified NRPSs, Tio12, Tio17, Tio18, Tio19, Tio20, Tio21, Tio22, Tio27 and Tio28. There are also several proteins which can be related to resistance processes, such as Tio5, Tio6, and Tio23. The possible thiocoraline pathway regulators identified in the sequences region correspond to Tio3, Tio4, Tio7, Tio24, Tio25. Finally, there are also several proteins related to the generation of the initiator unit 3-hydroxy-quinaldate, Tio8, Tio9, Tio10 and Tio11.

The genes, the gene interruption of which generates a phenotype that does not produce thiocoraline, are indicated in FIG. 1 by means of an asterisk (tio20, tio27 and tio28).

### Example 13

### Gene Interruption in tio28

For the purpose of demonstrating the involvement or not of the Tio28 protein in the biosynthesis of thiocoraline, the inactivation by gene interruption of the tio28 gene, and specifically of the single one of the adenylation domains it has, was carried out.

Two initiator oligonucleotides inside this adenylation domain (FL-T-102up and FL-T-102rp) were designed and used to amplify a 1,428 base pair area in tio28. The sequences of said initiator oligonucleotides are the following:

| | |
|---|---|
| FL-T-102up: | |
| 5′ -ACCTGAGGTACTGGGCGCAGC-3′ | (SEQ ID NO:45) |
| (21 nucleotides) | |
| FL-T-102rp: | |
| 5′ - CCGATCACCACCACCGTGGC-3′ | (SEQ ID NO:46) |
| (20 nucleotides) | |

The PCR program used was: 2 min at 94° C., 30 cycles (30 s at 94° C., 60 s at 53° C., 90 s at 68° C.), 5 min at 68° C. and 15 min at 4° C. The PCR reaction mixture contained: 1 μl of template DNA of cosmid pCT2c, 1 μl of each oligonucleotide at a 30 pmol/μl concentration, 7.5 μl of 2 mM dNTPs solution (dATP, dTTP, dCTP and dGTP), 1 μl of 50 mM MgSO₄, 5 μl of reaction buffer for Pfx (Invitrogene), 5 μl of Enhancer solution for Pfx (Invitrogene), 28 μl of distilled water and 0.5 μl of Pfx polymerase (Invitrogene).

The PCR product obtained, called PSV7, was cloned into the EcoRV site of plasmid pOJ260, generating pFL971.

The construct pFL971 was introduced in the conjugative E. coli ET12567 (pUB307) strain and from there, by conjugation, in the Micromonospora sp. ML1 strain. The transconjugant clones were selected with 25 μg/ml of apramycin and from the chromosomal DNA thereof, it was verified that the PSV7 region had indeed been interrupted by means of Southern hybridization. The probe used in this case was the PCR product PSV7.

The mutant Micromonospora sp. ΔPSV7 was grown in thiocoraline production medium MT4 and its mycelium was subsequently extracted with acetonitrile and analyzed by HPLC-MS (Example 5), giving as a result that this strain did not produce thiocoraline.

### Step 4. Heterologously Expressing Thiocoraline in Other Actinomycetes

To verify the involvement of the genes identified in the biosynthesis of thiocoraline, the heterologous expression of the cluster of thiocoraline genes in several Streptomyces species was assayed. The DNA region comprised between positions 1,393 (MseI restriction site) and 54,301 (AclI restriction site) of SEQ ID NO: 1 was chosen as the DNA fragment to be cloned into a plasmid replicative in E. coli and subsequently, into a plasmid replicative in E. coli/integrative in Streptomyces. This DNA region contains all the ORFs located between tio3 and tio28, both of them inclusive and complete (FIG. 1). The choice of this DNA region was due to the fact that the Tio3 and Tio28 proteins are the outermost proteins within the sequenced region which showed similarities with secondary metabolism proteins.

Due to its large size, said DNA region was obtained in steps, joining 3 independent DNA fragments which were obtained from 3 different cosmids (cosV33-D12, cosV19-B4 and pCT2c):
fragment A (20.2 kb): MseI (position 1,393 of SEQ ID NO:1)—NsiI (position 21,585 of SEQ ID NO:1);
fragment B (19 kb): NsiI (position 21,585 of SEQ ID NO:1)—EcoRI (position 40,636 of SEQ ID NO:1); and
fragment C (13.7 kb): EcoRI (position 40,636 of SEQ ID NO:1)—AclI (position 54,301 of SEQ ID NO:1).

To facilitate the subcloning, the complete DNA fragment was first subcloned into the plasmid replicative in E. coli pOJ260 (Example 14). The insert was rescued and subcloned into a vector replicative in E. coli/integrative of Streptomyces which contained the erythromycin resistance promoter (ermEp) (pARP) [Example 16] or without said promoter (pAR15AT) [Example 15]. This selected DNA region was cloned into said plasmids integrative of Streptomyces pAR15AT, in both directions (Example 17) and pARP (Example 18). Finally, said constructs were introduced in several streptomycetes by means of intergenus conjugation (Example 19).

### Example 14

### Cloning of the Selected DNA Region into E. coli Plasmid pOJ260

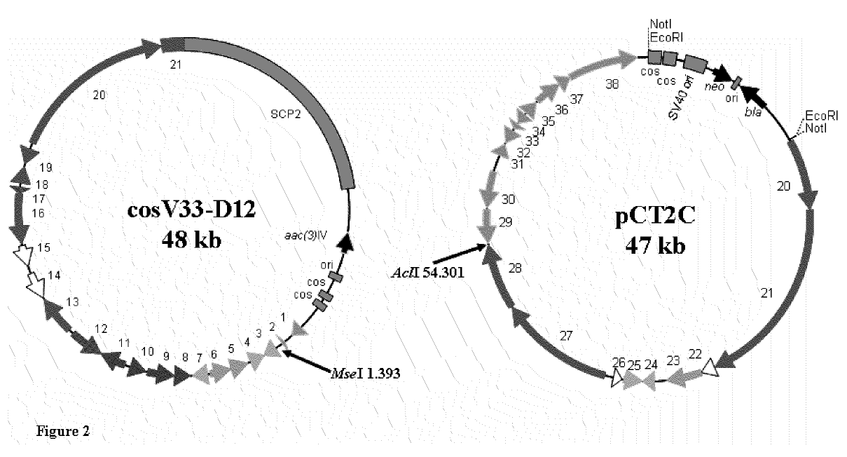

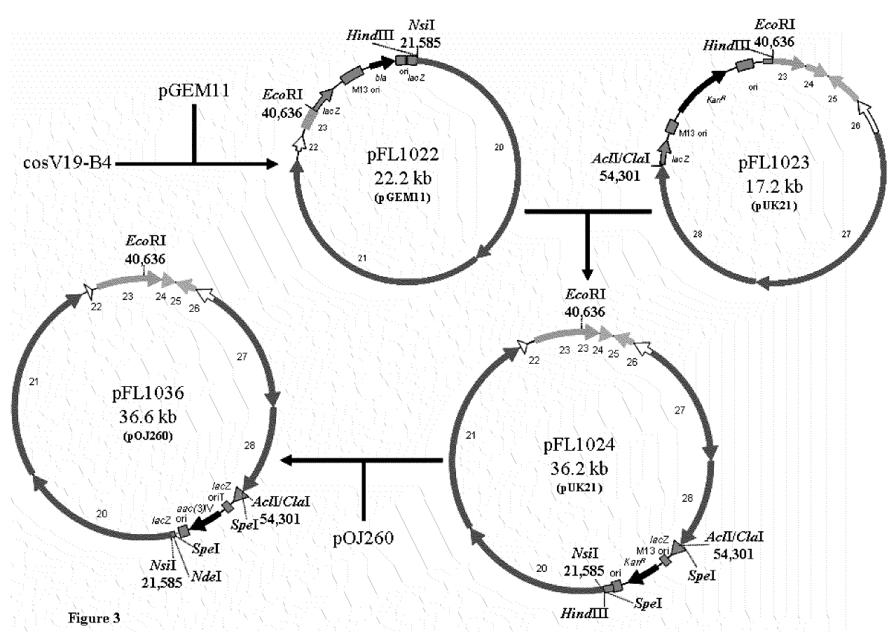

The DNA region located between the restriction sites EcoRI (position 40,636 of SEQ ID NO:1) and AcLI (position 54,301 of SEQ ID NO:1) was obtained from cosmid pCT2c (FIG. 2) by means of usual procedures (Sambrook et al. 2001). This DNA fragment was cloned into the unique restriction sites EcoRI and ClaI of E. coli plasmid pUK21 (Vieira et al. 1991, Gene 100, 189-194), generating the construct pFL1023 (FIG. 3).

The DNA region located between the restriction sites NsiI (position 21,585 of SEQ ID NO:1) and EcoRI (position 40,636 of SEQ ID NO:1) was obtained from cosmid cosV19-B4 by means of usual procedures (Sambrook et al. 2001). This DNA fragment was cloned into the unique restriction sites NsiI and EcoRI of E. coli plasmid pGEM-11Zf (Promega), generating the construct pFL1022 (FIG. 3).

These two DNA fragments were then joined. To that end, the DNA fragment located between the restriction sites NsiI (position 21,585 of SEQ ID NO:1) and EcoRI (position 40,636 of SEQ ID NO:1) present in pFL1022 was rescued by digesting with the restriction enzymes HindIII (located in the multiple cloning site immediately before the NsiI restriction site) and EcoRI. This fragment was then cloned into the unique restriction sites HindIII and EcoRI present in construct pFL1023, thus generating plasmid pFL1024 (FIG. 3).

The entire region cloned into pFL1024 was rescued as a SpeI band (thanks to these two restriction sites present at both ends of the multiple cloning site of pUK21) and cloned into the unique SpeI site of plasmid pOJ260, thus generating plasmid pFL1036 (FIG. 3).

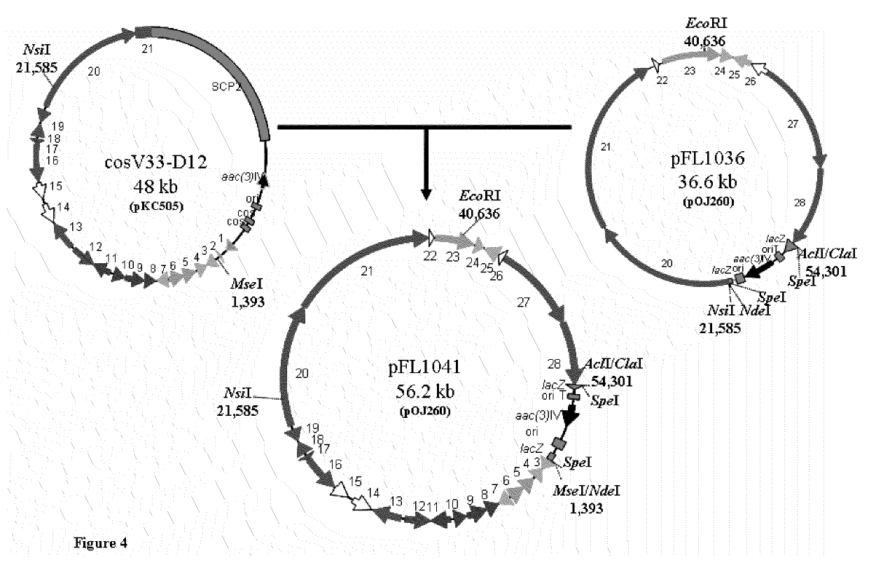

Finally, the fragment located between the cleavage sites MseI (position 1,393 of SEQ ID NO:1) and NsiI (position 21,585 of SEQ ID NO:1) was obtained from cosmid cosV33-D12 and it was cloned into the NdeI and NsiI sites, respectively, of pFL1036, generating construct pFL1041 (FIG. 4) containing in pOJ260 (Bierman et al. 1992, Gene 116, 43-49) the entire region comprised between the positions 1,393 (MseI restriction site) and 54,301 (AclI restriction site) of SEQ ID NO:1, i.e., from ORF tio3 to tio28, both of them inclusive and complete. Furthermore, in pFL1041, this region is flanked by two SpeI restriction sites. pFL1041 is a plasmid replicative in E. coli.

### Example 15

### Construction of the Plasmid Integrative of Streptomyces pAR15AT

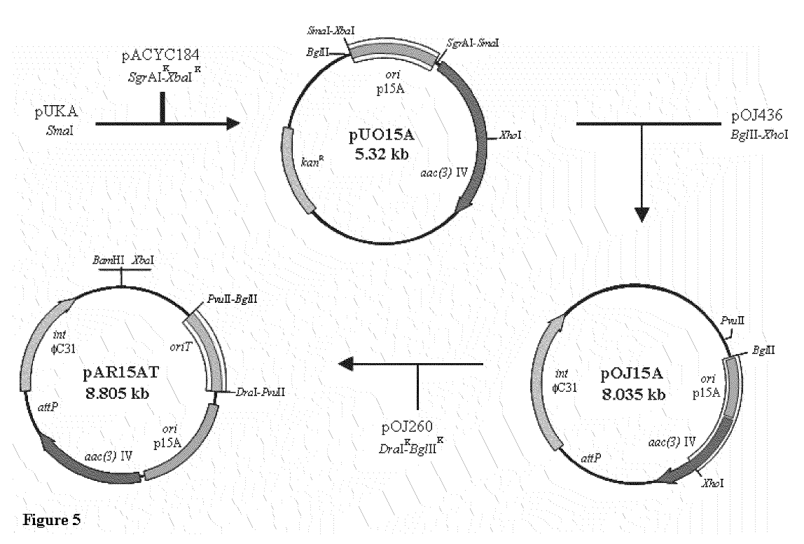

The replication origin of plasmid pACYC184 (Rose 1988, Nucleic Acids Res. 16, 355), ori p15A, was obtained as a SgrAI-XbaI fragment and was treated with the Klenow fragment of the E. coli DNA polymerase. This replication origin was cloned into the SmaI site of plasmid pUKA, thus obtaining plasmid pUO15A (FIG. 5). pUKA is a derivative of plasmid pUK21 (Vieira et al. 1991, Gene 100, 189-194) containing, cloned into its PstI-AccI restriction sites, the apramycin resistance gene obtained from cosmid pKC505 (Richardson at al. 1987, Gene 61, 231-241) as a PstI-EcoRI band.

A DNA fragment containing ori p15A next to the apramycin resistance gene aac(3)IV was obtained by means of a BglII-XhoI digestion on pUO15A. This fragment was cloned into plasmid pOJ436 using the same restriction enzymes (Bierman et al. 1992, Gene 116, 43-49), giving rise to construct pOJ15A (FIG. 5).

The DraI-BglII fragment (treated with Klenow) from plasmid pOJ260 and containing the conjugation origin oriT was cloned into the PvuII restriction site of pOJ15A. Plasmid pAR15AT is finally thus obtained (FIG. 5).

### Example 16

### Construction of the Plasmid Integrative of Streptomyces pARP

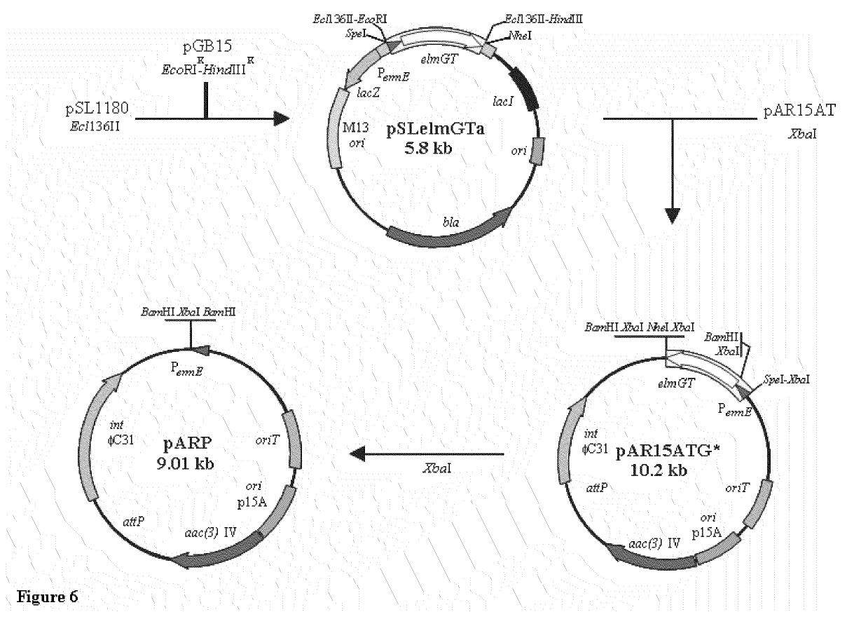

The elmGT glycosyltransferase gene from the elloramycin biosynthesis pathway, as a EcoRI-HindIII DNA fragment treated with Klenow obtained from plasmid pGB15 (Blanco et al. 2001, Chem. Biol. 8, 253-263), was cloned into the Ecl136II restriction site of pSL1180 (Amersham Pharmacia). Construct pSLelmGTa was thus obtained (FIG. 6), in which the elmGT gene is under the control of the constitutive ermE erythromycin resistance gene promoter (P_{ermE}).

A SpeI-NheI fragment obtained from pSLelmGTa which contained P_{ermE}-elmGT was cloned into the XbaI site of plasmid pAR15AT described in Example 15, obtaining construct pAR15ATG* (FIG. 6).

By means of XbaI digestion on plasmid pAR15ATG* and subsequent religation, the elmGT gene was eliminated, the P_{ermE} promoter being maintained, which gave rise to plasmid pARP (FIG. 6).

### Example 17

### Cloning of the Selected DNA Region into the Plasmid Integrative of Streptomyces pAR15AT, in Both Orientations

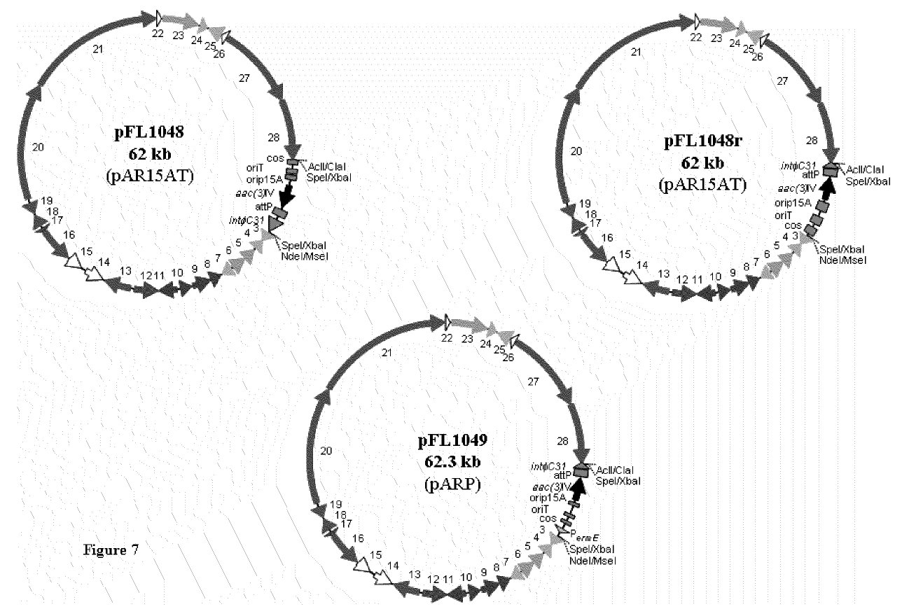

The SpeI DNA fragment from pFL1041 (FIG. 4) containing the region comprised between positions 1,393 (MseI restriction site) and 54,301 (AclI restriction site) of SEQ ID NO:1 was cloned, in both orientations, into the XbaI restriction site of plasmid pAR15AT (FIG. 5). Two new plasmids, called pFL1048 and pFL1048r (FIG. 7), with the apramycin resistance gene, replicative in E. coli and integrative in Streptomyces were thus generated by means of the system using the attP region of the φC31 phage.

### Example 18

### Cloning of the Selected DNA Region into the Plasmid Integrative of Streptomyces pARP after the ErmE Gene Promoter

In a similar way, the SpeI DNA fragment from pFL1041 (FIG. 4) containing the region comprised between positions 1,393 (MseI restriction site) and 54,301 (AclI restriction site) of SEQ ID NO:1 was cloned into the XbaI restriction site of plasmid pARP (FIG. 6). pFL1049 (FIG. 7) was thus generated, in which the ORF corresponding to tio3 (SEQ ID NO:4) is under the control of the constitutive promoter P_{ermE} present in pARP. This plasmid has the apramycin resistance gene, it is replicative in E. coli and integrative in Streptomyces by means of the system using the attP region of the φC31 phage.

### Example 19

### Heterologous Expression of the Thiocoraline Biosynthesis Pathway in Different Streptomycetes

Plasmid pFL1048 (FIG. 7) was introduced by conjugation from the E. coli ET12567 (pUB307) strain (Kieser et al. 2000) in the Streptomyces lividans TK21 (Kieser et al. 2000) and Streptomyces albus J1074 species (Chater et al. 1980, J. Gene. Microbiol. 116, 323-334).

Plasmid pFL1049 (FIG. 7) was introduced by conjugation from the E. coli ET12567 (pUB307) strain in the Streptomyces coelicolor M145 (Redenbach et al., 1996, Mol. Microbiol., 21, 77-96), Streptomyces lividans TK21, Streptomyces albus J1074 and Streptomyces avermitilis ATCC 31267 species.

Finally, plasmid pFL1048r (FIG. 7) was introduced by conjugation from the E. coli ET12567 strain (pUB307) in the Streptomyces lividans TK21 species.

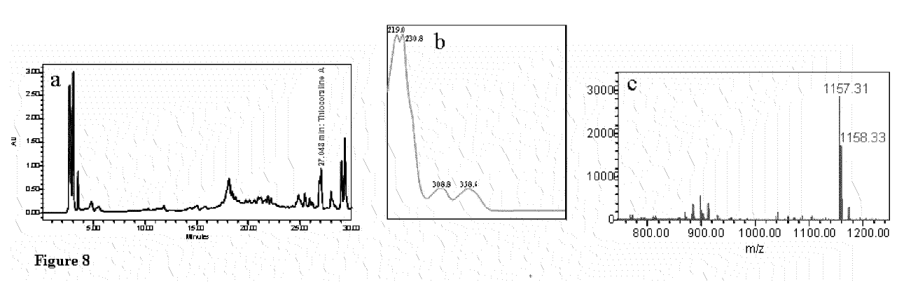

The results of the culture of the Streptomyces albus (pFL1049) clone in production medium R5A (Fernandez et al. 1998, J. Bacteriol. 180, 4929-4937) are shown in FIG. 8A. FIG. 8B shows the absorption spectrum of the peak with a retention time of 27 minutes in this chromatogram, and its mass spectrum (FIG. 8C), both of them being identical to those of purified thiocoraline.

1. An isolated nucleic acid molecule comprising a nucleotide sequence encoding at least one biosynthetic thiocoraline production pathway protein, or a biologically active fragment thereof.

2. A nucleic acid molecule according to claim 1, comprising a nucleotide sequence encoding all the biosynthetic thiocoraline production pathway proteins, or biologically active fragments thereof.

3. A nucleic acid molecule according to claim 1 or 2, comprising the nucleotide sequence shown in SEQ ID NO: 1 or its complementary strand.

4. A nucleic acid molecule hybridizing with the nucleic acid molecule of claim 3 and encoding at least one biosynthetic thiocoraline production pathway protein, or a biologically active fragment thereof.

5. A nucleic acid molecule according to claim 1, comprising a nucleotide sequence encoding a biosynthetic thiocoraline production pathway protein, or a biologically active fragment thereof.

6. A nucleic acid molecule according to claim 5, selected from the group consisting of:
the nucleic acid molecule comprising nucleotides 2-535 of SEQ ID NO: 1 (orf1);
the nucleic acid molecule comprising nucleotides 993-1130c of SEQ ID NO: 1 (orf2);
the nucleic acid molecule comprising nucleotides 1517-2131 of SEQ ID NO: 1 (tio3);
the nucleic acid molecule comprising nucleotides 2154-2822c of SEQ ID NO: 1 (tio4);
the nucleic acid molecule comprising nucleotides 2970-3791c of SEQ ID NO: 1 (tio5);
the nucleic acid molecule comprising nucleotides 3794-4777c of SEQ ID NO: 1 (tio6);
the nucleic acid molecule comprising nucleotides 4904-5611 of SEQ ID NO: 1 (tio7);
the nucleic acid molecule comprising nucleotides 5701-6426c of SEQ ID NO: 1 (tio8);
the nucleic acid molecule comprising nucleotides 6426-7688c of SEQ ID NO: 1 (tio9);
the nucleic acid molecule comprising nucleotides 7733-8524c of SEQ ID NO: 1 (tio10);
the nucleic acid molecule comprising nucleotides 8791-10002 of SEQ ID NO: 1 (tio11);
the nucleic acid molecule comprising nucleotides 10002-11590c of SEQ ID NO: 1 (tio12);
the nucleic acid molecule comprising nucleotides 11847-13634 of SEQ ID NO: 1 (tio13);
the nucleic acid molecule comprising nucleotides 13734-15005c of SEQ ID NO: 1 (tio14);
the nucleic acid molecule comprising nucleotides 15005-16354c of SEQ ID NO: 1 (tio15);
the nucleic acid molecule comprising nucleotides 16441-18744c of SEQ ID NO: 1 (tio16);
the nucleic acid molecule comprising nucleotides 18774-19055c of SEQ ID NO: 1 (tio17);
the nucleic acid molecule comprising nucleotides 19260-20036 of SEQ ID NO: 1 (tio18);
the nucleic acid molecule comprising nucleotides 20146-20880c of SEQ ID NO: 1 (tio19);
the nucleic acid molecule comprising nucleotides 21188-28969 of SEQ ID NO: 1 (tio20);
the nucleic acid molecule comprising nucleotides 28979-38398 of SEQ ID NO: 1 (tio21);
the nucleic acid molecule comprising nucleotides 38449-38661 of SEQ ID NO: 1 (tio22);
the nucleic acid molecule comprising nucleotides 38642-41263 of SEQ ID NO: 1 (tio23);
the nucleic acid molecule comprising nucleotides 41835-42368 of SEQ ID NO: 1 (tio24);
the nucleic acid molecule comprising nucleotides 42395-43255c of SEQ ID NO: 1 (tio25);
the nucleic acid molecule comprising nucleotides 43340-43741c of SEQ ID NO: 1 (tio26);
the nucleic acid molecule comprising nucleotides 44152-49563 of SEQ ID NO: 1 (tio27);
the nucleic acid molecule comprising nucleotides 49635-53669 of SEQ ID NO: 1 (tio28);
the nucleic acid molecule comprising nucleotides 53749-55305c of SEQ ID NO: 1 (orf29);
the nucleic acid molecule comprising nucleotides 55384-57222c of SEQ ID NO: 1 (orf30);
the nucleic acid molecule comprising nucleotides 57895-58467c of SEQ ID NO: 1 (orf31);
the nucleic acid molecule comprising nucleotides 58535-59206c of SEQ ID NO: 1 (orf32);
the nucleic acid molecule comprising nucleotides 59298-59564c of SEQ ID NO: 1 (orf33);
the nucleic acid molecule comprising nucleotides 59611-60114c of SEQ ID NO: 1 (orf34);
the nucleic acid molecule comprising nucleotides 60202-60888 of SEQ ID NO: 1 (orf35);
the nucleic acid molecule comprising nucleotides 60960-62240 of SEQ ID NO: 1 (orf36);
the nucleic acid molecule comprising nucleotides 62300-62833 of SEQ ID NO: 1 (orf37);
the nucleic acid molecule comprising nucleotides 62925-64650 of SEQ ID NO: 1 (orf38); or
fragments thereof encoding biologically active fragments of biosynthetic thiocoraline production pathway proteins.

7. A nucleic acid molecule according to claim 1, comprising a nucleotide sequence encoding two or more biosynthetic thiocoraline production pathway proteins, or biologically active fragments thereof.

8. A nucleic acid molecule according to claim 7, comprising two or more genes selected from the genes identified as orf1, orf2, tio3, tio4, tio5, tio6, tio7, tio8, tio9, tio10, tio11, tio12, tio13, tio14, tio15, tio16, tio17, tio18, tio19, tio20, tio21, tio22, tio23, tio24, tio25, tio26, tio27, tio28, orf29, orf30, orf31, orf32, orf33, orf34, orf35, orf36, orf37, orf38 and fragments thereof encoding biologically active fragments of biosynthetic thiocoraline production pathway proteins.

9. A nucleic acid molecule according to claim 1, comprising a nucleotide sequence encoding at least one biosynthetic thiocoraline production pathway protein, or a biologically active fragment thereof, or a mutant or variant thereof, wherein said protein is selected from the group consisting of the proteins identified as ORF1 (SEQ ID NO: 2), ORF2 (SEQ ID NO: 3), Tio3 (SEQ ID NO: 4), Tio4 (SEQ ID NO: 5), Tio5 (SEQ ID NO: 6), Tio6 (SEQ ID NO: 7), Tio7 (SEQ ID NO: 8), Tio8 (SEQ ID NO: 9), Tio9 (SEQ ID NO: 10), Tio10 (SEQ ID NO: 11), Tio11 (SEQ ID NO: 12), Tio12 (SEQ ID NO: 13), Tio13 (SEQ ID NO: 14), Tio14 (SEQ ID NO: 15), Tio15 (SEQ ID NO: 16), Tio16 (SEQ ID NO: 17), Tio17 (SEQ ID NO: 18), Tio18 (SEQ ID NO: 19), Tio19 (SEQ ID NO: 20), Tio20 (SEQ ID NO: 21), Tio21 (SEQ ID NO: 22), Tio22 (SEQ ID NO: 23), Tio23 (SEQ ID NO: 24), Tio24 (SEQ ID NO: 25), Tio25 (SEQ ID NO: 26), Tio26 (SEQ ID NO: 27), Tio27 (SEQ ID NO: 28), Tio28 (SEQ ID NO: 29), ORF29 (SEQ ID NO: 30), ORF30 (SEQ ID NO: 31), ORF31 (SEQ ID NO: 32), ORF32 (SEQ ID NO: 33), ORF33 (SEQ ID NO: 34), ORF34 (SEQ ID NO: 35), ORF35 (SEQ ID NO: 36), ORF36 (SEQ ID NO: 37), ORF37 (SEQ ID NO: 38), ORF38 (SEQ ID NO: 39) and combinations thereof.

10. A nucleic acid molecule according to claim 1, comprising a nucleotide sequence comprising an orfs selected from the group consisting of orf1, orf2, tio3, tio4, tio5, tio6, tio7, tio8, tio9, tio10, tio11, tio12, tio13, tio14, tio15, tio16, tio17, tio18, tio19, tio20, tio21, tio22, tio23, tio24, tio25, tio26, tio27, tio28, orf29, orf30, orf31, orf32, orf33, orf34, orf35, orf36, orf37, orf38 and combinations thereof, or of the corresponding regions, mutants or variants thereof.

11. A nucleic acid molecule according to claim 1, isolated from Micromonospora sp.

12. A composition comprising at least one nucleic acid molecule according to any of claims 1 to 11.

13. A probe comprising a nucleic acid molecule according to any of claims 1 to 11 or a fragment thereof.

14. A vector comprising a nucleic acid molecule according to any of claims 1 to 11 or a composition according to claim 12.

15. A host cell transformed or transfected with a vector of the invention.

16. A host cell according to claim 15, wherein said host cell is a microorganism, preferably a bacterium.

17. A host cell according to claim 16, wherein said bacterium is a Gram-positive bacterium, preferably an actinomycete or a streptomycete.

18. A protein encoded by the nucleic acid molecule of the invention.

19. A protein according to claim 18, selected from the group consisting of the proteins identified as ORF1 (SEQ ID NO: 2), ORF2 (SEQ ID NO: 3), Tio3 (SEQ ID NO: 4), Tio4 (SEQ ID NO: 5), Tio5 (SEQ ID NO: 6), Tio6 (SEQ ID NO: 7), Tio7 (SEQ ID NO: 8), Tio8 (SEQ ID NO: 9), Tio9 (SEQ ID NO: 10), Tio10 (SEQ ID NO: 11), Tio11 (SEQ ID NO: 12), Tio12 (SEQ ID NO: 13), Tio13 (SEQ ID NO: 14), Tio14 (SEQ ID NO: 15), Tio15 (SEQ ID NO: 16), Tio16 (SEQ ID NO: 17), Tio17 (SEQ ID NO: 18), Tio18 (SEQ ID NO: 19), Tio19 (SEQ ID NO: 20), Tio20 (SEQ ID NO: 21), Tio21 (SEQ ID NO: 22), Tio22 (SEQ ID NO: 23), Tio23 (SEQ ID NO: 24), Tio24 (SEQ ID NO: 25), Tio25 (SEQ ID NO: 26), Tio26 (SEQ ID NO: 27), Tio27 (SEQ ID NO: 28), Tio28 (SEQ ID NO: 29), ORF29 (SEQ ID NO: 30), ORF30 (SEQ ID NO: 31), ORF31 (SEQ ID NO: 32), ORF32 (SEQ ID NO: 33), ORF33 (SEQ ID NO: 34), ORF34 (SEQ ID NO: 35), ORF35 (SEQ ID NO: 36), ORF36 (SEQ ID NO: 37), ORF37 (SEQ ID NO: 38), ORF38 (SEQ ID NO: 39), and combinations thereof, or biologically active fragments thereof.

20. A process for producing a protein involved in the biosynthesis of thiocoraline according to any of claims 18 or 19, which comprises growing, under suitable conditions, a thiocoraline-producing organism, and, if desired, isolating one or more of said proteins involved in the biosynthesis of thiocoraline.

21. A method for producing thiocoraline which comprises growing, under suitable conditions for producing said compound, a thiocoraline-producing organism in which the number of copies of genes encoding proteins involved in the biosynthesis of thiocoraline has been increased, and, if desired, isolating thiocoraline.

22. A method for producing thiocoraline which comprises growing, under suitable conditions for producing said compound, a thiocoraline-producing organism in which the expression of the genes encoding the proteins responsible for the biosynthesis of thiocoraline has been modulated by means of manipulating or substituting one or more genes encoding proteins involved in the biosynthesis of thiocoraline or by means of manipulating the sequences responsible for regulating the expression of said genes, and, if desired, isolating thiocoraline.

23. A method according to any of claims 21 or 22, wherein said thiocoraline-producing organism is an actinomycete, preferably Micromonospora sp.

24. A method for producing thiocoraline which comprises growing, under suitable conditions for producing said compound, a host cell according to any of claims 15 to 17, and, if desired, isolating thiocoraline.

25. A method according to claim 24, wherein said host cell is an actinomycete or a streptomycete.

26. A process, based on the use of genes responsible for the biosynthesis of thiocoraline from Micromonospora sp. ML1, for the production of said compound in another actinomycete, comprising:
(1) obtaining mutants affected in specific genes of the thiocoraline biosynthesis pathway;
(2) isolating the Micromonospora sp. ML1 chromosome region containing the cluster of genes responsible for the biosynthesis of thiocoraline;
(3) obtaining and analyzing the nucleotide sequence of the cluster of genes responsible for the biosynthesis of thiocoraline; and
(4) heterologously producing thiocoraline in other actinomycetes.
